(19) United States
(12) Patent Application Publication
(10) Pub. No.: US 2026/0045372 A1
(43) Pub. Date: Feb. 12, 2026

(54) Methods and Systems to Provide Enhanced Network Slice-Based Communications Between Healthcare Workers for Patient Care and Documentation

(71) Applicant: T-Mobile Innovations LLC, Overland Park, KS (US)

(72) Inventors: Christina CLAURE, Menlo Park, CA (US); Joao TEIXEIRA, Overland Park, KS (US)

(21) Appl. No.: 18/796,213

(22) Filed: Aug. 6, 2024

(51) Int. Cl. G16H 80/00; G16H 10/60; G16H 40/67

(52) U.S. Cl. CPC G16H 80/00; G16H 10/60; G16H 40/67

(57) ABSTRACT

A method comprises determining, by an authentication application, whether a first device is permitted to communicate with a second device using one or more healthcare-dedicated network slices based on at least one of a first device identifier identifying the first device or a second device identifier identifying the second device, and determining, by the authentication application, a network slice of the one or more healthcare-dedicated network slices in the communication network based on one or more network attributes associated with the network slice and the medical data when the first device is permitted to communicate with the second device using the one or more healthcare-dedicated network slices.

### CROSS-REFERENCE TO RELATED APPLICATIONS

None.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

The recent global pandemic has led to nursing shortages worldwide. The actual cause of the nursing shortage is not necessarily the lack of nurses available to work. Still, the senior nurses are increasingly hesitant to work in conditions during and following the pandemic. Meanwhile, junior nurses are available and ready to work but need guidance from more senior nurses to correctly be trained. Junior nurses may also need more knowledge to provide optimal patient care and efficiently operate medical equipment. Therefore, the need for more senior nurses with sufficient experience is causing various problems in the healthcare industry.

### SUMMARY

In an embodiment, a method implemented in a communication network to provide enhanced, network slice-based communications between healthcare workers for patient care and documentation is disclosed. The method comprises receiving, by an aggregator application executing at a healthcare facility system in the communication network, current patient data associated with a patient from a first device operated by a first healthcare worker and medical device data associated with the patient from one or more medical devices, and transmitting, by the aggregator application, a first device identifier, second device identifier, and medical data comprising at least one of the current patient data or the medical device data to an authentication application executing at an authentication system in the communication network, in which the first device identifier uniquely identifies the first device, and wherein the second device identifier uniquely identifies a second device operated by a second healthcare worker. The method further comprises determining, by the authentication application, that the first device is permitted to communicate with the second device using one or more healthcare-dedicated network slices in the communication network based on at least one of the first device identifier or the second device identifier, identifying, by the authentication application, a network slice of the one or more healthcare-dedicated network slices based on the medical data, and transmitting, by the authentication application to the aggregator application, network slice data describing the network slice identified for the first device to communicate with the second device. The method further comprises encrypting, by the aggregator application, the medical data based on the second device identifier to obtain encrypted medical data, packaging, by the aggregator application, the encrypted medical data with the network slice data for transmission through a network-slice specific path within the communication network to the second device, and decrypting, by an application at the second device, the encrypted medical data using the first device identifier.

In another embodiment, a method implemented in a communication network to provide artificial intelligence enhanced communications between healthcare workers for patient care and documentation is disclosed. The method comprises receiving, by an authentication application executing at an authentication system in the communication network, from a first device operated by a first healthcare worker, a request to transmit medical data associated with a patient along one or more healthcare-dedicated network slices in the communication network to a second device operated by a second healthcare worker, and determining, by the authentication application, whether the first device is permitted to communicate with the second device using the one or more healthcare-dedicated network slices based on at least one of a first device identifier identifying the first device or a second device identifier identifying the second device, in which the first device is positioned proximate to a patient while the second device is positioned at least a predefined distance away from the patient. The method further comprises determining, by the authentication application, a network slice of the one or more healthcare-dedicated network slices in the communication network based on one or more network attributes associated with the network slice and the medical data when the first device is permitted to communicate with the second device using the one or more healthcare-dedicated network slices, and transmitting, by the authentication application to the first device, network slice data describing at least one of the network slice or a path in the network slice identified for the first device to communicate with the second device. The method further comprises encrypting, by a first application executing at the first device, the medical data based on the second device identifier to obtain encrypted medical data, transmitting, by the first application, the encrypted medical data with the network slice data through the network slice within the communication network to the second device, and decrypting, by a second application executing at the second device, the encrypted medical data using the first device identifier.

In yet another embodiment, a system is disclosed. The system comprises a non-transitory memory, a processor coupled to the non-transitory memory, and an authentication application stored at the non-transitory memory. The authentication application, when executed by the processor, causes the processor to be configured to receive, from a first device operated by a first healthcare worker, a request to transmit medical data associated with a patient along one or more healthcare-dedicated network slices in a communication network to a second device operated by a second healthcare worker, determine whether the first device is permitted to communicate with the second device using the one or more healthcare-dedicated network slices based on at least one of a first device identifier identifying the first device or a second device identifier identifying the second device, in which the first device is positioned proximate to a patient while the second device is positioned at least a predefined distance away from the patient, determine, based on a network slice policy, a network slice of the one or more healthcare-dedicated network slices when the first device is permitted to communicate with the second device using the one or more healthcare-dedicated network slices, wherein the network slice policy indicates that the medical data is to be transmitted using the network slice based on one or more network attributes associated with the network slice, and transmit, to the first device, network slice data describing at least one of the network slice or a path in the network slice identified for the first device to communicate with the second device.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 is a block diagram of a communication network according to various embodiments of the disclosure.

FIG. 2 is a block diagram illustrating an embodiment for authenticating a healthcare worker device to use a healthcare network slice in the communication network of FIG. 1 according to various embodiments of the disclosure.

FIGS. 3A, 3B, and 3C, are block diagrams illustrating embodiments for providing enhanced, network slice-based communications between healthcare workers and patients in the communication network of FIG. 1 according to various embodiments of the disclosure.

FIG. 4 is a flowchart of a first method for providing enhanced, network slice-based communications between healthcare workers and patients according to various embodiments of the disclosure.

FIG. 5 is a flowchart of a second method for providing enhanced, network slice-based communications between healthcare workers and patients according to various embodiments of the disclosure.

FIGS. 6A and 6B are block diagrams illustrating a communication system similar to the communication system of FIG. 1 according to an embodiment of the disclosure.

FIG. 7 is a block diagram of a computer system implemented within the communication system of FIG. 1 according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

U.S. Patent App. 18/788,098 [related 4900-10700] (hereinafter referred to as the “AI Communications Application”) introduces the concept of leveraging internal private networks and/or cellular networks with artificial intelligence (AI) models to enable intelligent and secure communications between junior healthcare workers (e.g., junior nurses) that work directly with the patients and remotely positioned senior healthcare workers (e.g., senior nurses). The AI Communications Applications is hereby incorporated by reference in its entirety.

As described in the AI Communications Application, a healthcare facility system includes a routing application, a record application, and a medical application, each of which may, in some cases, use an AI model to generate various types of recommendations based on medical data pertaining to a patient. The medical data is received from one or more devices operated by a junior healthcare worker and medical devices collecting data from the patient. For example, the recommendations may include record recommendations relating to suggested documentation to add to a patient record of the patient, medical recommendations associated with the medical care/diagnosis/treatment of the patient, step-by-step instructions for operating medical equipment concerning the patient, and/or additional medical training directly relevant to the patient care. The recommendations may be sent to a second device operated by the remotely positioned senior healthcare worker, such that the senior healthcare worker can use the second device to confirm/reject recommendations as needed. The senior healthcare worker may send the confirmed recommendations to the first device to assist the junior healthcare worker in providing patient care while offering equipment instructions and general medical training to the junior healthcare worker.

Therefore, the communications channel connecting the medical devices, the first devices operated by the junior healthcare worker, the second devices operated by the senior healthcare workers, the AI model hosting system, and the healthcare facility system are used for the transmission of highly sensitive patient data (e.g., including personally identifiable information (PII)), medical diagnostic/treatment data, and/or other types of healthcare-related data). As such, securing these communications is significant from the perspective of maintaining the security of the data traffic, but also significant in view of complying with various government/industry regulations and standards. Moreover, the healthcare data transmitted between these entities may route through the same paths and routing configurations as general, non-healthcare data traffic (i.e., healthcare data may not be given a higher priority over general data traffic). In addition, the network may not be configured to route healthcare data along a path with more optimal network resources (e.g., a path with higher bandwidth, lower latency, lower jitter, etc.). Therefore, the communication channels between healthcare workers may experience various technical problems related to the lack of data security and the inefficient and ineffective use of network resources while routing highly sensitive healthcare data.

The present disclosure addresses the foregoing technical problems by providing a technical solution in the technical field of communication systems, particularly those used in the healthcare industry. The embodiments disclosed herein are directed to managing communication channels between the first devices operated by the junior healthcare worker, the second devices operated by the senior healthcare workers, the AI model hosting system, and the healthcare facility system. In an embodiment, the communication channels may be provisioned in a network based on one or more healthcare-dedicated network slices. The network elements in the network slices are programmed with security mechanisms for securing healthcare data traffic, as further described herein. The healthcare-dedicated network slices may also be programmed to forward the healthcare data traffic along slice-specific paths with optimal network attributes (e.g., low latency, high bandwidth, etc.). The embodiments disclosed herein are also directed to providing additional layers of security to the healthcare data traffic by first authenticating the devices that are requesting the use of the healthcare network slices and then encrypting/decrypting the healthcare data traffic based on identification data of a receiving device/system, as further described herein. Therefore, the embodiments disclosed herein address the aforementioned technical problems by providing several layers of security to the healthcare data traffic, and ensuring that the healthcare data traffic is sent along network slices with optimal network attributes, such that healthcare data traffic is sent in a prioritized, efficient, and secure manner.

As mentioned above and described in the AI Communications Application, the embodiments disclosed herein may be implemented in a communication network including a healthcare facility system, one or more first devices operated by junior healthcare workers (also referred to herein as “first healthcare workers”), one or more second devices operated by senior healthcare workers (also referred to herein as “second healthcare workers”), one or more medical devices collecting data (e.g., biometric data/vital signs/images) from the patient, and an AI model (e.g., hosted by a computer system). The communications network may include network elements (e.g., routers, switches, virtual network functions (VNFs), etc.) that are configured to forward data across healthcare network slices according to various data routing protocols, forwarding rules, and network slice data included in the incoming healthcare data traffic. As described herein, the communication network may also include a data store maintaining information related to the healthcare network slices. The communication network may also include an authentication system, which may be used to authenticate the devices communicating using the healthcare network slices.

A network slice may refer to a virtualized, isolated portion of a network infrastructure, providing a specific set of resources and services tailored to meet the requirements of particular user groups, applications, or services. For example, within a 5G core network, network slices allow for the creation of multiple virtualized network instances, each optimized to meet the diverse requirements of different use cases, providing flexibility, scalability, and efficient management of network elements to provide 5G core network services. In the embodiments disclosed herein, the communication network may include network elements configured to forward traffic along network slices to meet the requirements of the respective network slice. The configured network slices may include healthcare-dedicated network slices that meet the network requirements (e.g., bandwidth, latency, jitter, etc. requirements) and security requirements (e.g., minimum security parameters for transmitting healthcare-related data) for a given type of healthcare data (e.g., video data, emergency medical data, patient identification data, etc.) or based on a source of the healthcare data (e.g., first device, second device, AI model, etc.).

Each network slice may be associated with a network profile stored in a data store in the communication network. A network profile may include an identifier of the associated network slice and data indicating the network attributes and security of each associated network slice (e.g., security mechanisms, quality of service, service level agreements, resource allocation, security policies, traffic management, service dependencies, etc.). Different types of healthcare data (e.g., video data, emergency medical data, patient identification data, etc.) may be associated with varying attributes of network/requirements (e.g., bandwidth allocation, maximum latency, maximum allowable packet loss rate, reliability level, etc.) and/or different security attributes/requirements (e.g., isolation/segmentation mechanisms, access control, intrusion detection/prevention, authentication, authorization, network traffic monitoring, etc.). Similarly, different healthcare data sources may be associated with varying attributes of network/requirements and/or security attributes/requirements. The data store may maintain network policies defining, for example, the healthcare network slice for different types of healthcare data based on the network attributes/requirements and/or security attributes/requirements for the different types of healthcare data. Similarly, the network policies may also define the healthcare network slice that is to be used for data originating from particular sources based on the network attributes/requirements and/or security attributes/requirements for the different sources.

In an embodiment, the healthcare network slices may be pre-configured, existing network slices that may each be associated with predefined network attributes/requirements and/or security attributes/requirements, and thus may be used for healthcare-related data transmission. In this embodiment, network elements in the communication network may prioritize healthcare data transmissions through an existing network slice over other, non-healthcare-related data transmissions. For example, non-healthcare-related data transmissions may be dropped or delayed to account for incoming healthcare data transmissions, to prioritize the transmission of emergency healthcare data.

In another embodiment, the healthcare network slices may be newly provisioned in the communication network (e.g., by configuring the network elements in the communication network according to the network attributes/requirements and/or security attributes/requirements for each healthcare network slice). For example, the healthcare network slices may include reserved network elements and resources dedicated solely to transmitting healthcare data. In this embodiment, certain types of healthcare data may get prioritized over other types of healthcare data, for example, based on the inclusion of an emergency flag in the healthcare data. In this way, the communication network may include healthcare network slices, which prioritize transmitting healthcare data through the network while enforcing predefined security mechanisms during the transmission of the data and guaranteeing that the data is transmitted according to baseline network attributes/requirements.

The authentication system may include an authentication application and a data store indicating authenticated devices (e.g., first devices of junior healthcare workers, second devices of senior healthcare workers, medical devices, etc.) that are permitted to forward healthcare data using a healthcare network slice. In particular, the data store may store device identifiers (or system identifiers) identifying the devices (or systems) that have pre-registered with the authentication system. During the pre-registration process, the devices (or systems) may have provided evidence to the authentication system that the respective device is indeed used for healthcare data, medical data, and/or patient data communications, such that the authentication system performs an evidence-based verification and registration of the device. Upon registration, an identifier of the device (or system) may be added to a list of authenticated device identifiers identifying registered devices. The device identifiers may be, for example, an identifier of a Subscriber Identity Module (SIM) at the respective device, in which the SIM may refer to a physical SIM card or an electronic SIM profile, each storing data used to authenticate with and use telecommunications carrier network resources. As another example, the device identifiers may be an address of the device (e.g., Internet Protocol address). The device identifiers may be any other identifier or value uniquely identifying a device. The authentication application may first use the authenticated device identifiers to determine whether a device is permitted to a healthcare network slice before determining which healthcare network slice may be used for healthcare data communications from the device, as further described herein.

As mentioned above and in the AI Communications Application, a junior healthcare worker may be working directly with a patient and operating a first device (e.g., computer or tablet) to obtain current patient data describing the current symptoms experienced by the patient. For example, the first device may be a computing device embodied as a wearable lapel, including a microphone and a radio transceiver, which may capture a recording or text dictation of the conversation between the junior healthcare worker and the patient. Another first device may be a computer or tablet, and the junior healthcare worker may manually enter current patient data describing the current symptoms experienced by the patient into the first device via a user interface. Meanwhile, as the junior healthcare worker examines the patient, one or more medical devices may be hooked up to the patient, taking images of the patient or otherwise collecting medical device data associated with the patient. For example, the medical devices may include a camera, a sensor, a cardiac monitor, a defibrillator, an oxygen delivery system, a computed tomography scanner, a suction unit, airway management equipment, a splinting and immobilization device, first aid supplies, intravenous supplies, diagnostic equipment and/or various types of equipment. The medical device data obtained from the different medical devices may include, for example, biometric information and/or vital signs (e.g., heart rate, blood pressure, respiratory rate, temperature).

Each of the first devices operated by the junior healthcare worker and the medical devices may include a radio transceiver for cellular radio communications across the communication network. When the junior healthcare worker and the patient are located at a healthcare facility (e.g., hospital, urgent care center, or other medical care location), the associated healthcare facility system may include an aggregator application. The aggregator application may obtain medical data related to the patient (e.g., the current patient data from the first device and the medical device data from the medical devices), an identifier identifying the first device (e.g., SIM identifier), an identifier identifying the medical devices, and/or an identifier identifying a destination of the medical data (e.g., an identifier of the second device operated by a senior healthcare worker). The identifiers of the first device, second device, medical devices, healthcare facility system, system hosting the AI model, etc., may be stored in a data store securely accessible by the aggregator application or may be received from another application (e.g., routing application, record application, medical application, etc.) at the healthcare facility system.

The aggregator application may transmit the medical data and the identifiers of the devices to the authentication application of the authentication system, for example, in a request to use a healthcare network slice to forward the medical data to an identified destination. For example, the request may include a source identifier (e.g., identifier of the first device/identifier of the medical device), a destination identifier (e.g., identifier of the second device/identifier of the healthcare facility), the medical data (e.g., or an indication of the type of medical data), and an indication that the request is to use a healthcare network slice.

As mentioned above, the aggregator application aggregates the medical data/identifiers and transmits the request to the authentication application on behalf of the first healthcare worker when the first healthcare worker and the patient are positioned in a healthcare facility having a private network. However, in cases when the first healthcare worker and patient are not positioned in the healthcare facility, but instead are positioned remotely (e.g., at the patient home) and/or external to a healthcare facility, an application executing at the first device may perform the same aforementioned steps performed by the aggregator application. The application at the first device may obtain the current patient data from the first healthcare worker (e.g., provided via a user interface of the first device), receive medical device data from medical devices collecting data from the patient, package the current patient data and medical device data into medical data, and transmit the medical data with the first device identifier and the second device identifier to the authentication application.

The authentication application may receive the medical data, the first device identifier, and the second device identifier from the aggregator application of the healthcare facility system or the application of the first device. The authentication application may first determine whether the first device/aggregator application at the healthcare facility system is permitted to communicate with the second device using one or more healthcare network slices based on whether the first device identifier, healthcare facility system identifier, and/or second device identifier are authenticated device identifiers (i.e., identifying devices that have pre-registered as sending healthcare data and thus are authorized to use the healthcare network slices).

When the first device/aggregator application at the healthcare facility system is permitted to communicate with the second device using a healthcare network slice, the authentication application may then use the network policies to identify a network profile that indicates a network slice having the network attributes and security attributes that meet the requirements for transmitting the medical data received from the first device. As described above, a network policy may identify a network slice for a source of the medical data or the type of medical data sent by the first device. In some cases, different data items in the medical data may be associated with different network profiles and thus different network slices. The authentication application may obtain the network slice data of each identified network profile and transmit the network slice data back to the first device.

The first device may receive the network slice data from the authentication application and obtain the second device identifier of the second device when the destination of the medical data is the second device (or obtain an identifier of another healthcare facility system or other destination based on the destination of the medical data). The first device may encrypt the medical data using the second device identifier (or identifier of the destination) as the encryption key based on an encryption algorithm to obtain encrypted medical data. For example, the second device identifier may be a SIM identifier of a SIM card of the second device, and the encryption may be performed using the SIM identifier.

The first device may generate one or more packets, including the medical data and the network slice data. The network slice data may be added to the packets (e.g., in a header of one or more packets including the medical data as a payload or as metadata to the one or more packets carrying the medical data). In this way, when network elements in the network receive the packets, the network elements may use the network slice data to identify a slice-specific path within the network slice along which to forward the packets.

The second device (or destination) may receive the packets containing the encrypted medical data. The second device may obtain the first device identifier, for example, from a data store accessible by the second device or from the healthcare facility system. The second device may decrypt the encrypted medical data using the first device identifier, identifying the first device as the key for decryption based on a decryption algorithm. For example, the first device identifier may be a SIM identifier of an eSIM profile at the first device, and the decryption may be performed using the SIM identifier. In this way, encryption and decryption of medical data is performed based on a device identifier of the other party in communication.

The second device may confirm recommendations for transmission back to the first device, and may otherwise maintain a line of communication with the first device as the first healthcare worker is caring for the patient, as described in the AI Communications Application. The second device may encrypt the confirmed recommendations and other data using the first device identifier, and generate one or more packets with the confirmed recommendations/other data and the network slice data allocated for healthcare communications between the first device and the second device. Again, the network elements are configured to forward the packets along a slice-specific path in the network slice based on the network slice data carried in the packets. The first device may receive the packets and decrypt the recommendations/other data using the second device identifier.

In this way, the embodiments disclosed herein add three layers of security to the transmission of healthcare data across the network: the first layer being the use of the healthcare-dedicated network slice, the second layer being the device identifier based authentication to use the healthcare network slice, and the third layer being the destination identifier based encryption/decryption of all healthcare data transmitted through the healthcare network slice. Accordingly, the embodiments disclosed herein serve to create secure and tailored radio communication channels between a senior, remote healthcare worker, one or more junior healthcare workers with direct access to a patient, medical devices, and a healthcare facility system. The disclosed communication channels utilize dedicated healthcare network slices that are provisioned based on network attributes/requirements and security attributes/requirements for different types of healthcare data and different entities sending/receiving the healthcare data. Moreover, pre-registered devices are authenticated before being allowed access to a healthcare network slice, and data traffic flowing through a healthcare network slice may be additionally secured with destination-based encryption/decryption schemes. Therefore, in general, the embodiments disclosed herein also serve to increase healthcare system capacity by increasing security, prioritizing healthcare-related emergency communications, while providing a network connection for senior healthcare workers to train junior healthcare workers while patient care.

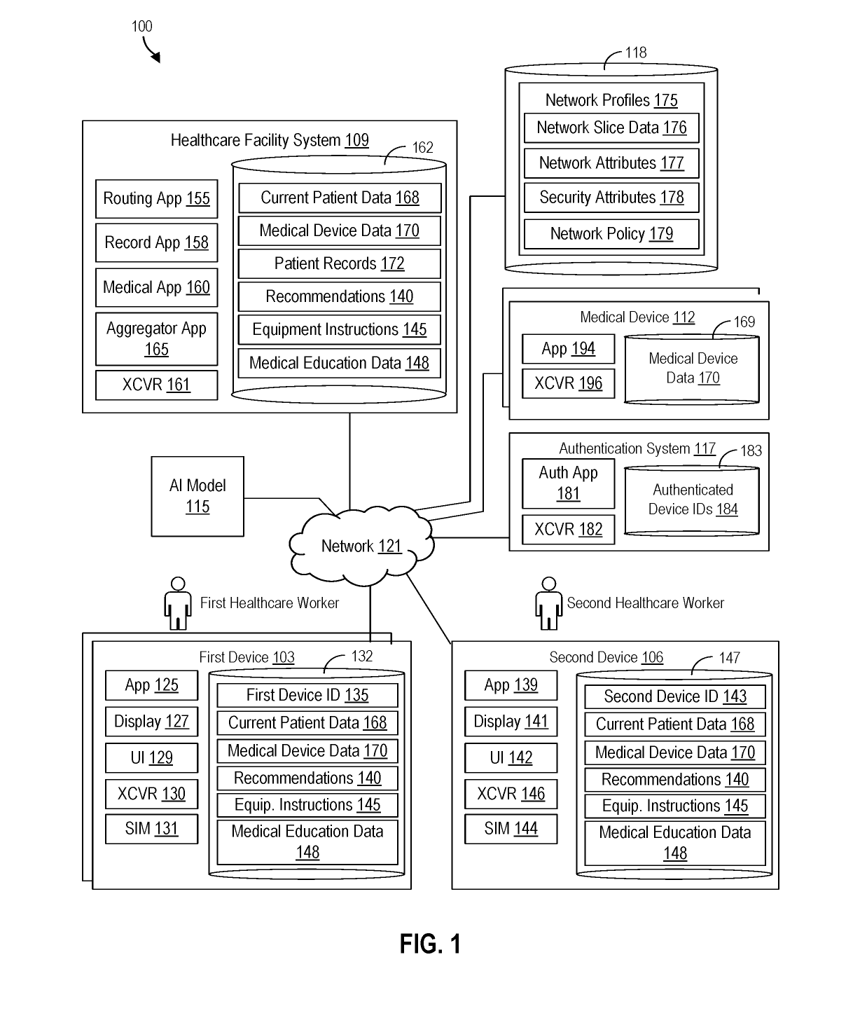

Turning now to FIG. 1, a communication network 100 is described. The communication network 100 includes one or more first devices 103, one or more second devices 106, a healthcare facility system 109, one or more medical devices 112, an AI model 115, an authentication system 117, a data store 118, and a network 121. Network 121 may be one or more private networks, one or more public networks, or a combination thereof, interconnecting the devices 103, 106, healthcare facility system 109, medical devices 112, AI model 115, authentication system 117, and data store 118. While FIG. 1 illustrates the healthcare facility system 109, AI model 115, authentication system 117, and data store 118 as being separate from the network 121, it should be appreciated that in some embodiments, the healthcare facility system 109, AI model 115, authentication system 117, and data store 118 may be part of the network 121. While FIG. 1 illustrates the healthcare facility system 109 as separate from the data store 118 and authentication system 117, it should be appreciated that in some embodiments, the healthcare facility system 109 may include the data store 118 and/or the authentication system 117. While FIG. 1 illustrates the authentication system 117 as separate from the data store 118, it should be appreciated that in some embodiments, the authentication system 117 may include the data store 118. While FIG. 1 illustrates the AI model 115 as separate from the healthcare facility system 109, it should be appreciated that in some embodiments, the AI model 115 may be included as part of the healthcare facility system 109.

The first device 103 may be operated by a junior healthcare worker (also referred to herein as a “first healthcare worker”). As mentioned above, the junior healthcare worker may have less experience than senior healthcare workers, and may work directly with the patient to provide patient care based on instructions/recommendations provided by the senior healthcare workers. The first device 103 may be, for example, a mobile phone, tablet, personal computer, wearable device, or any other device that includes one or more components such as a display 127, a user interface 129, a radio transceiver 130 (shown in FIG. 1 as “XCVR 130”), a SIM 131, a microphone, a speaker, a camera, a processor, a memory, etc. The radio transceiver 130 may be a cellular transceiver configured to establish a wireless communication link with a cell site in the communication network 100 according to a 5G, a long-term evolution (LTE), a code division multiple access (CDMA), or a global system for mobile communication (GSM) telecommunication protocol. The radio transceiver 130 may also support relatively short-range radio communication, and for example, may be embodied as a WiFi radio transceiver, a Bluetooth radio transceiver, or another short-range radio transceiver. The SIM 131 may refer to at least one of a physical SIM card or eSIM profile, each including the data and credentials used to authenticate with a telecommunications carrier network. Each SIM 131 may be associated with an identifier, which may be a value uniquely identifying the physical SIM card or eSIM profile of the first device 103.

The junior healthcare worker may use multiple different first devices 103 simultaneously. For example, the junior healthcare worker may be wearing a wearable first device 103 (e.g., watch or lapel), which may include a camera and a microphone, and may operate a computer (e.g., another first device 103) positioned within a patient room simultaneously.

For example, the first device 103 may be implemented as a computer system 700. The first device 103 may include an application 125 for receiving various types of data directly from the junior healthcare worker (e.g., via the user interface 129), from the medical devices 112, or even from other external data stores, and sending this data to the appropriate entity through a healthcare network slice, if permitted. To this end, the first device 103 may include a data store 132 (e.g., one or more memories) for storing different types of data associated with the first device 103, collected by the first device 103, and/or received from other external devices/systems. As shown in FIG. 1, the data store 132 may store a first device identifier 135, current patient data 168, medical device data 170, recommendations 140, equipment instructions 145, and medical education data 148. The first device identifier 135 may be a value uniquely identifying the first device 103. For example, the first device identifier 135 may be an identifier of the SIM 131 of the first device 103. The current patient data 168 may include data describing a current state or condition of the patient as recorded by the junior healthcare worker (e.g., symptoms currently experienced by the patient, patient identification information, etc.) For example, the current patient data 168 may include data (e.g., recording or text) of a conversation between the junior healthcare worker and the patient. The medical device data 170 may include data received from the medical devices 112 positioned with respect to the patient, in some cases, hooked up to the patient or performing a diagnostic procedure on the patient. For example, the medical device data 170 may include biometric information and/or vital signs (e.g., heart rate, blood pressure, respiratory rate, temperature).

Recommendations 140 may refer to the confirmed recommendations (e.g., medical recommendations, record recommendations, etc.) generated using the AI model 115 and confirmed by the senior healthcare worker. The equipment instructions 145 may refer to the (second healthcare worker confirmed) step-by-step instructions, settings, or configurations that may be generated using the AI model 115 and presented to the junior healthcare worker to assist in using various types of medical equipment concerning a patient. The medical education data 148 may refer to (second healthcare worker confirmed) patient-specific medical education or training information, which may be received from an external education-related data store and/or the AI model 115, and may in some cases be presented to the junior healthcare worker to assist in patient care.

The second device 106 may be operated by a senior healthcare worker (also referred to herein as a “second healthcare worker”) who is positioned remotely from the junior healthcare worker and the patient (e.g., at least a predefined distance from the junior healthcare worker). For example, the senior healthcare worker may be positioned in a different room or office within a healthcare facility or external to the healthcare facility (e.g., at home). The second device 106 may be, for example, a mobile phone, tablet, personal computer, or any other device that includes one or more components such as a display 141, a user interface 142, a radio transceiver 146 (shown in FIG. 1 as “XCVR 146”), a SIM 144, a microphone, a speaker, a camera, a processor, a memory, etc. The radio transceiver 146 may be configured to establish a wireless communication link with a cell site in the communication network 100 according to a 5G, a LTE, a CDMA, or a GSM telecommunication protocol. The radio transceiver 146 may also support relatively short-range radio communication, and for example, may be embodied as a WiFi radio transceiver, a Bluetooth radio transceiver, or another short-range radio transceiver. The SIM 144 may refer to at least one physical SIM card or eSIM profile, each including the data and credentials used to authenticate with a telecommunications carrier network. Each SIM 144 may be associated with an identifier, which may be a value uniquely identifying the physical SIM card or eSIM profile of the second device 106.

For example, the second device 106 may be implemented as a computer system 700. The second device 106 may include an application 139 for receiving various types of data from the healthcare facility system 109 and other sources in the communication network 100. To this end, the second device 106 may include a data store 147 (e.g., one or more memories) for storing different types of data associated with the second device 106 or received from other external devices/systems. As shown in FIG. 1, the data store 147 may store a second device identifier 143, the current patient data 168, the medical device data 170, the recommendations 140, the equipment instructions 145, and the medical education data 148. The second device identifier 143 may be a value uniquely identifying the second device 106. For example, the second device identifier 143 may be an identifier of the SIM 144 of the second device 106. The current patient data 168 may include data indicative of current symptoms experienced by the patient, biometric data of the patient, and/or vital signs of the patient. The medical device data 170 may include biometric data and/or vital signs of the patient collected from the medical devices 112. The recommendations 140 may refer to the recommendations (e.g., medical recommendations, record recommendations, etc.) generated by the routing application 155, the record application 158, and/or the medical application 160 using the AI model 115, and sent to the second device 106. The equipment instructions 145 may refer to the step-by-step instructions, settings, or configurations that may be generated using the AI model 115, and sent to the second device 106. The medical education data 148 may refer to patient-specific medical education or training information, which may be received from an external education-related data store or the AI model 115.

The healthcare facility system 109 may be a computer system, server software/hardware, or a collection of processors, memories, and/or networking resources, used to manage, receive, and transmit different types of data as described herein. For example, each healthcare facility system 109 may be embodied as a cloud-based system, which may include one or more data stores and memories located together or separately across geographically disparate locations, separate from the respective healthcare facility or group of healthcare workers. Each healthcare facility system 109 may also be embodied as a local set of data stores and memories positioned within or proximate to a respective healthcare facility. A healthcare facility may be, for example, a hospital, an emergency department, trauma center, cardiac center, stroke center, maternity hospital, psychiatric hospital, rehabilitation center, specialty hospital, urgent care center, long-term care facility, etc. A single healthcare facility may employ multiple different groups of healthcare workers, each contracted with a separate organization. Nevertheless, the healthcare facility system 109 may maintain data related to multiple different groups of healthcare workers.

The healthcare facility system 109 may include a routing application 155, a record application 158, a medical application 160, an aggregator application 165, a radio transceiver 161, and a data store 162. The radio transceiver 161 may be a cellular transceiver configured to establish a wireless communication link with a cell site in the communication network 100 according to a 5G, a LTE, a CDMA, or a GSM telecommunication protocol. The routing application 155, record application 158, and medical application 160 may each be instructions stored across one or more memories, which may be executed by a processor of the healthcare facility system 109 to perform the steps described herein. The routing application 155 may dynamically connect one or more junior healthcare workers (i.e., first devices 103) to an optimal senior healthcare worker (e.g., second device 106), as described in the AI Communications Application. The record application 158 may use the data received by the first device 103 and from the medical devices 112 to generate and store patient records 172, as described in the AI Communications Application. The medical application 160 may generate recommendations 140, equipment instructions 145, and/or medical education data 148 using the AI model 115 based on various types of data, as described in the AI Communications Application. The aggregator application 165 may aggregate the data from various sources (e.g., first devices 103, second devices 106, medical devices 112, etc.) within an associated healthcare facility, and then package, encrypt/decrypt, send/receive, and store the data in the data store 162.

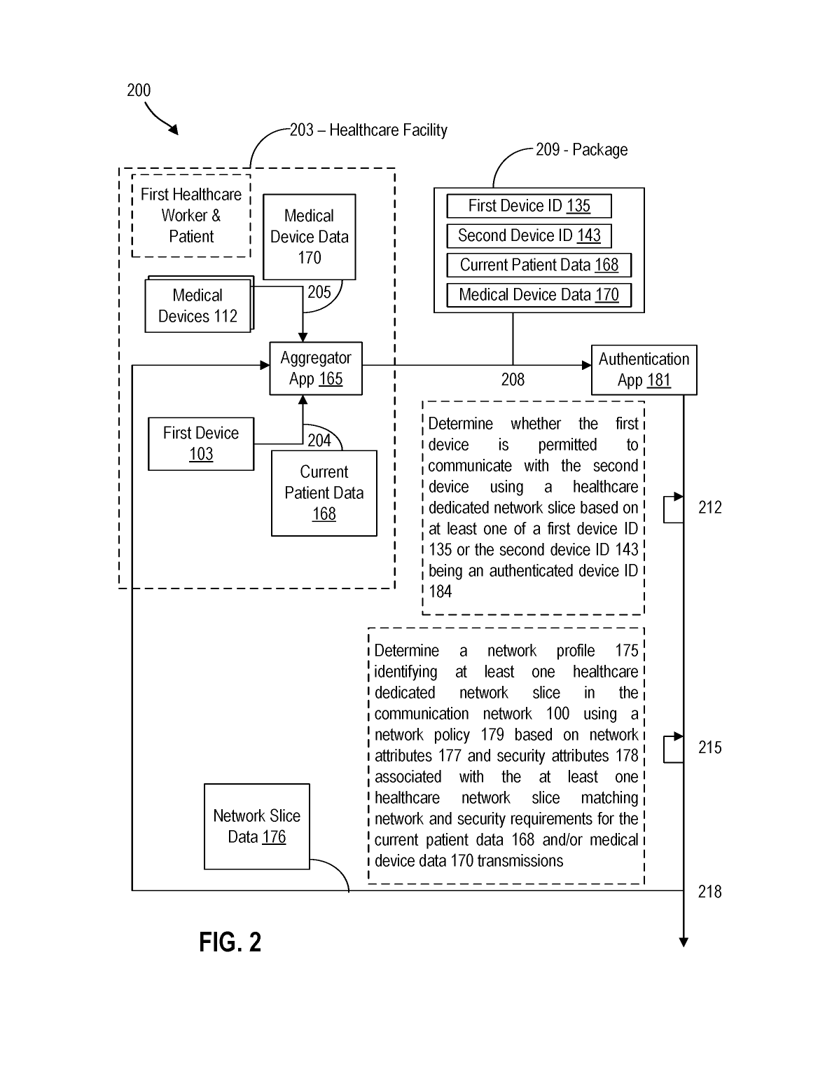

The data store 162 may be a collection of one or more memories (distributed or co-located) for storing various types of data. While the data store 162 is shown in FIG. 2 as being part of the healthcare facility system 109, it should be appreciated that the data store 162 may be external to the healthcare facility system 109. The data store 162 may store data collected from the first devices 103 and the medical devices 112. For example, the data store 162 may store the current patient data 168, which as described above includes data describing a current state or condition of a patient. The data store 162 may also store medical device data 170 received from one or more medical devices 112 via a radio connection.

The data store 162 may also store patient records 172, which may be received from the second device 106. The patient records 172 may include various types of documented data associated with a patient. For example, a patient record 172 for a patient may include comprehensive patient demographics, medical history, clinical assessments, diagnosis and treatment plans, progress notes detailing the patient's condition and response to treatment, nursing care plans outlining interventions and monitoring, medication administration records, consents/legal documents, discharge planning details, and communication logs among healthcare providers. The data store 162 may also store the recommendations 140 generated by the record application 158 and/or the medical application 160 using the AI model 115 and historical data, the equipment instructions 145 generated by the medical application 160 using the AI model 115, and the medical education data 148 generated by the medical application 160 using the AI model 115.

The medical devices 112 refer to medical equipment, tools, or devices, which may be used to collect biometric data of a patient, vital signs of a patient, and/or data describing a current state or condition of the patient. For example, the medical devices 112 may include a camera, a sensor, a cardiac monitor, a defibrillator, an oxygen delivery system, a computed tomography scanner, a suction unit, airway management equipment, a splinting and immobilization device, first aid supplies, intravenous supplies, diagnostic equipment and/or various types of equipment. The medical devices 112 may each include an application 194 for collecting/processing medical device data 170 and a radio transceiver 196 for transmitting the medical device data 170 to the healthcare facility system 109/second device 106. The radio transceiver 196 may be a cellular transceiver configured to establish a wireless communication link with a cell site in the communication network 100 according to a 5G, a LTE, a CDMA, or a GSM telecommunication protocol. The radio transceiver 196 may also support relatively short-range radio communication, and for example, may be embodied as a WiFi radio transceiver, a Bluetooth radio transceiver, or another short-range radio transceiver. The medical device data 170 may include, for example, biometrics, vital signs, scanned images, X-ray images, blood test results, cardiac readings, etc., each indicative of a current medical condition of the patient.

The authentication system 117 may be a computer system, server software/hardware, or a collection of processors, memories, and/or networking resources, used to manage, receive, and transmit different types of data as described herein. The authentication system 117 may include an authentication application 181, a radio transceiver 182, and a data store 183 indicating authenticated devices (e.g., first devices 103 of junior healthcare workers, second devices 106 of senior healthcare workers, medical devices 112, healthcare facility systems 109, etc.) that are permitted to forward healthcare data using a dedicated healthcare network slice. In particular, the data store 183 may store authenticated device identifiers 184 identifying the devices that have pre-registered with the authentication system 117, in which the pre-registration process may involve evidence-based verification that the respective device is indeed used for healthcare data, medical data, and/or patient data communications. The authenticated device identifiers 184 may be, for example, an identifier of a SIM 131, 144 at the respective device, in which the SIM may refer to a physical SIM card or an electronic SIM profile. As another example, the authenticated device identifier 184 may be an address of the device (e.g., Internet Protocol address). The authenticated device identifiers 184 may alternatively be any other identifier or value uniquely identifying a device. The authentication application 181 may use the authenticated device identifiers 184 to first determine whether a device is permitted to access or use a healthcare network slice, before determining which healthcare network slice may be used for healthcare data communications from the device, using the data stored at the data store 118.

The data store 118 may be a collection of one or more memories (distributed or co-located) for storing data related to the healthcare network slices provisioned in the communication network. The data store 118 may store network profiles 175 each respectively describing a healthcare network slice. The network profile 175 may store the network attributes 177 (e.g., bandwidth allocation, maximum latency, maximum allowable packet loss rate, reliability level, etc.) of a network slice. The network profile may store the security attributes 178 (e.g., isolation/segmentation mechanisms, access control, intrusion detection/prevention, authentication, authorization, network traffic monitoring, etc.) of a network slice. The network profile 175 may also store the network policy 179, defining a rule that the authentication application 181 may use to identify a network policy 179 for data traffic received from a source. For example, the network policy 179 may define mappings between certain types of healthcare data, healthcare data originating from a source device, and/or healthcare data destined for a destination device and the corresponding network profile 175 identifying a network slice. The network slice data 176 may include at least one of an identifier of the corresponding network slice, the network attributes 177, the security attributes 178, and/or instructions for network elements in the network slice to ensure forwarding the healthcare data along the network slice.

The AI model 115 may be a computer system (e.g., including both software and hardware components) designed to make predictions or forecasts (e.g., the medical recommendations 150 and/or record recommendations) based on patterns or trends learned from historical data (e.g., historical medical data 180 and historical medical device data 193). The AI model 115 may be implemented using software (e.g., algorithms, logic, and code) stored across memories. The host of the AI model 115 (which may be an external server or the healthcare facility system 109) may provide the computational resources to execute the AI model 115. AI model 115 may be implemented as one or more different types of models using, for example, linear regression, decision trees, support vector machines, neural networks, or ensemble methods. The AI model 115 may be a machine learning model, deep learning model, neural networking model, natural language processing (NLP) model, learning action model, or any other type of AI model. It should be appreciated that any AI/predictive model may be used, and the underlying algorithms, computations, and machine learning libraries used by the AI model 115 should not be limited herein. Additional details on the AI model 115 are included in the AI Communications Application.

Turning now to FIG. 2, shown is a block diagram illustrating an example method 200 for authenticating a healthcare worker device to use a healthcare network slice in the communication network 100. The method 200 may be performed by the first device 103, medical devices 112, the aggregator application 165 at the healthcare facility system 109, and the authentication application 181 and the authentication system 117. In the example shown in FIG. 2, the first healthcare worker and the patient are located in a healthcare facility 203. Thus, the first device(s) 103 and medical device(s) 112 are also located in the healthcare facility 203. The healthcare facility 203 is associated with the healthcare facility system 109, which includes the aggregator application 165. In another embodiment, the first healthcare worker and the patient may be located separate from a healthcare facility 203 (e.g., at a home of the patient). In such a case, application 125 of the first device 103 may perform the same steps as the aggregator application 165 in method 200.

At operation 204, the aggregator application 165 may receive the current patient data 168 from one or more first devices 103. For example, the first device 103 may use the radio transceiver 130 to transmit the current patient data 168 at one time, periodically at random or according to a predefined schedule, or continuously, and the radio transceiver 161 at the healthcare facility system 109 may receive the current patient data 168 for storage at the data store 162. Similarly, at operation 205, the aggregator application 165 may receive the medical device data 170 from one or more medical devices 112. For example, the medical devices 112 may use the radio transceiver 196 to transmit the medical device data 170 at one time, periodically at random or according to a predefined schedule, or continuously, and the radio transceiver 161 at the healthcare facility system 109 may receive the medical device data 170 for storage at the data store 162. In another embodiment in which the first healthcare worker and the patient are located external to the healthcare facility system 109, the application 125 at the first device 103 may obtain (e.g., receive) the current patient data 168 and medical device data 170.

At operation 208, the aggregator application 165 may generate a package 209 (e.g., one or more data packets) including the first device identifier 135 identifying the first device 103 (or an identifier of the healthcare facility system 109), a second device identifier 143 identifying the second device 106, the current patient data 168, and the medical device data 170. The aggregator application 165 may have received the second device identifier 143 from the routing application 155 after the routing application 155 identified the optimal senior healthcare worker and the second device 106 of the senior healthcare worker to which to connect to the junior healthcare worker and the first device 103, as described in the AI Communications Application. The aggregator application 165 may have otherwise received the first device identifier 135 (or an identifier of the healthcare facility system 109) and/or the second device identifier 143 from a data store accessible by the aggregator application 165. The aggregator application 165 may transmit the package 209 to the authentication application 181. In another embodiment in which the first healthcare worker and the patient are located external to the healthcare facility system 109, the application 125 at the first device 103 may generate and send the package 209 to the authentication application 181.

At operation 212, the authentication application 181 may determine whether the first device 103 (and/or the aggregator application 165 at the healthcare facility system 109) is permitted to communicate with the second device 106 using a healthcare-dedicated network slice based on at least one of the first device identifier 135 (or an identifier of the healthcare facility system 109) or the second device identifier 143 being an authenticated device identifier 184 stored in the data store 183. For example, both endpoints of healthcare communications (e.g., the first device 103 of the junior healthcare worker/the healthcare facility system 109 and the second device 106 of the senior healthcare worker) may need to have pre-registered with the authentication system 117 to have permission to use a healthcare network slice for healthcare data communications.

When the first device 103 is permitted to communicate with the second device 106, method 200 may proceed to operation 215. At operation 215, the authentication application 181 may determine a network profile 175 identifying at least one healthcare network slice in the communication network 100. The authentication application 181 may identify the at least one healthcare network slice using a network policy 179 based on network attributes 177 and/or security attributes 178 of the at least one healthcare network slice that matches the network and security requirements for the current patient data 168 and/or medical device data 170 transmissions. For example, the network policy 179 may indicate a mapping or association between the predefined requirements for the current patient data 168 and/or medical device data 170 transmissions and the network attributes 177 and security attributes 178 of at least one healthcare network slice.

At operation 218, the authentication application 181 may obtain the network slice data 176 from the network profile 175 and transmit the network slice data 176 to the aggregator application 165. In another embodiment where the first healthcare worker and the patient are located external to the healthcare facility system 109, the authentication application 181 may transmit the network slice data 176 to the first device 103.

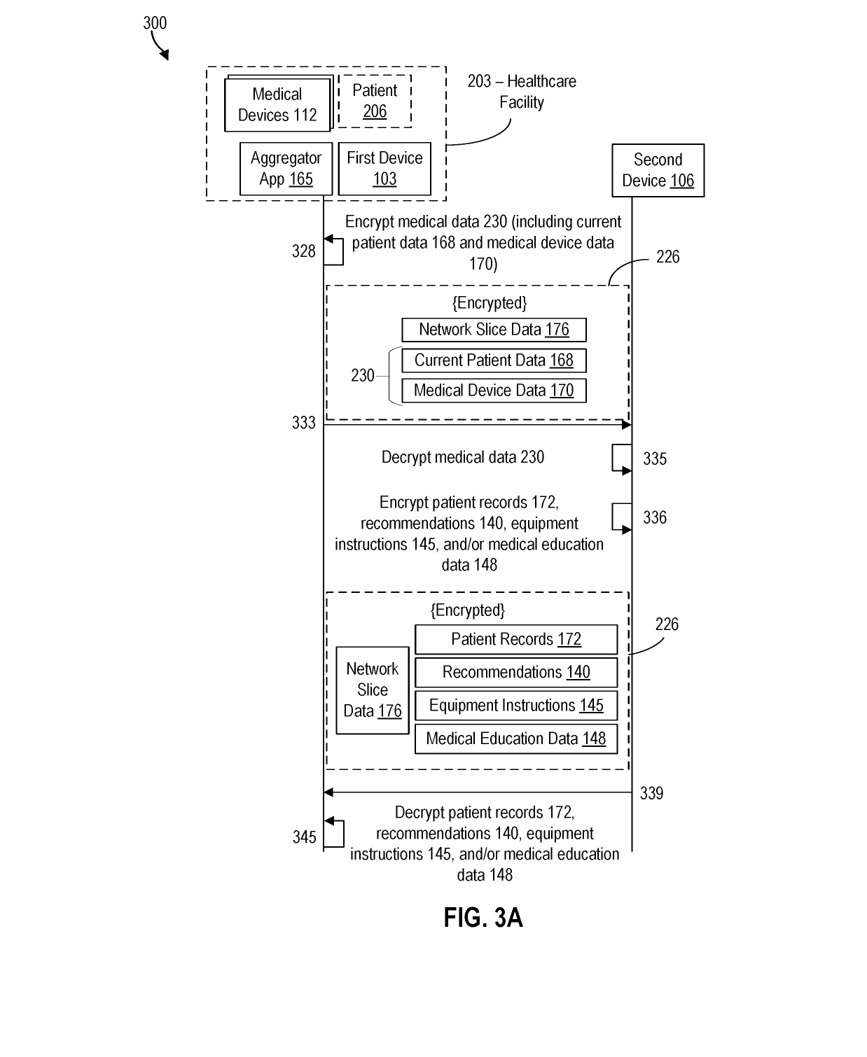

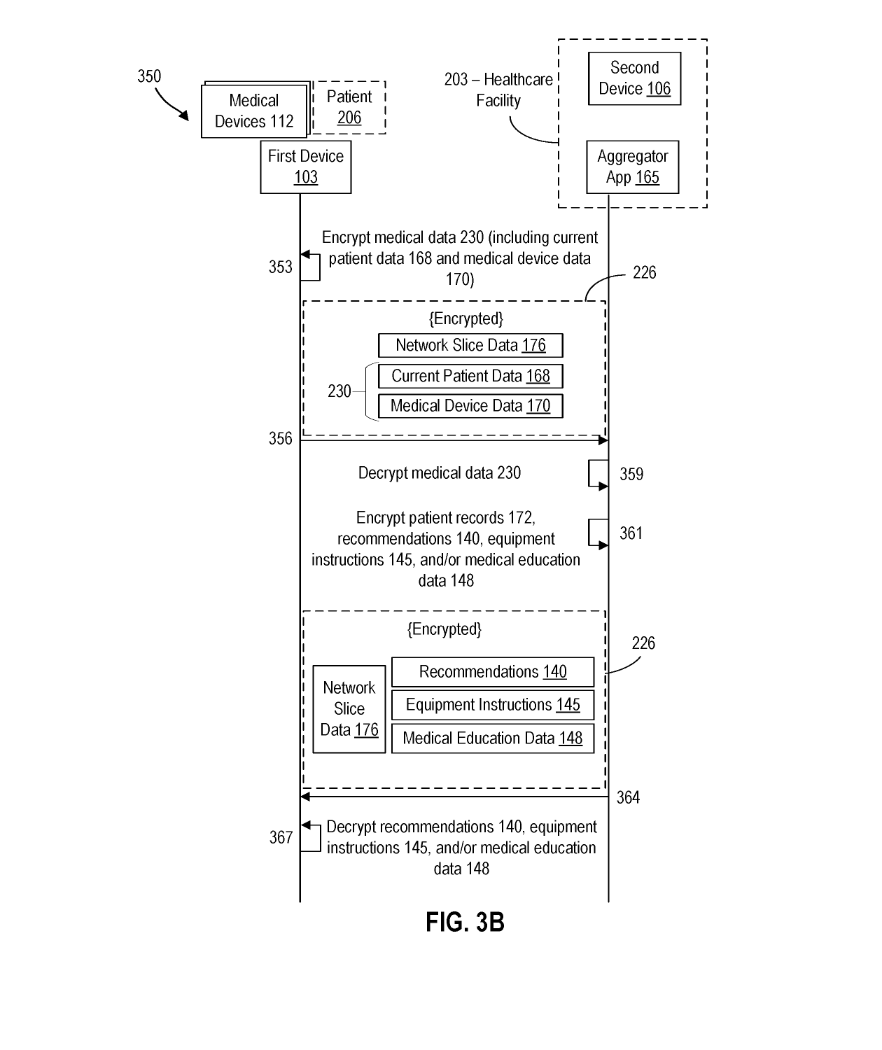

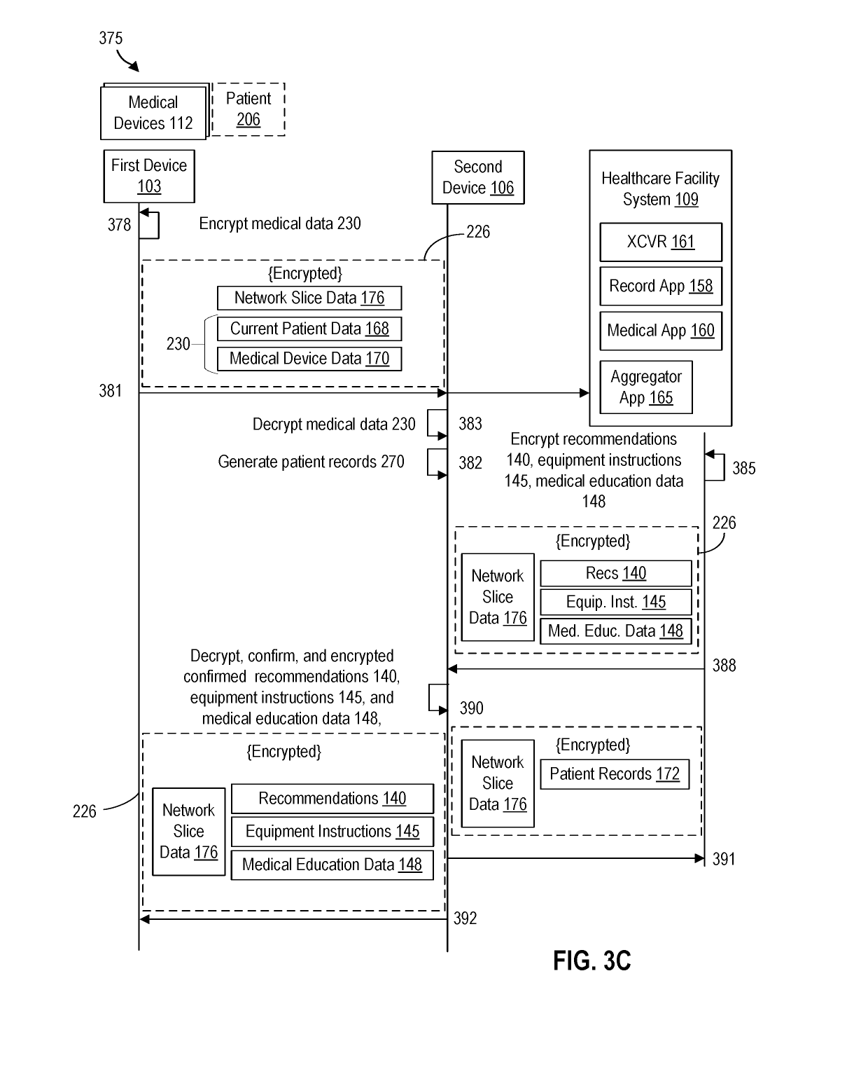

Turning now to FIGS. 3A-3C, shown are block diagrams illustrating embodiments for providing enhanced, network slice-based communications between healthcare workers and patients. Specifically, FIG. 3A illustrates a situation in which the patient and a junior healthcare worker are located in a healthcare facility 203 while the senior healthcare worker is located external to/outside the healthcare facility 203, FIG. 3B illustrates a situation in which the senior healthcare worker is located in a healthcare facility 203 while the patient and the junior healthcare worker are located external to the healthcare facility 203, and FIG. 3C illustrates a situation in which the junior healthcare worker, the patient, and the senior healthcare worker are all located external to the healthcare facility 203.

FIG. 3A is a block diagram illustrating a method 300 for providing enhanced, network slice-based communications between healthcare workers and patients, in which the patient and the junior healthcare worker are located in a healthcare facility 203 while the senior healthcare worker is located external to the healthcare facility 203. In the example shown in FIG. 3A, the first device(s) 103 and medical device(s) 112 are located in the healthcare facility 203 with the first healthcare worker and the patient. The healthcare facility 203 is associated with the healthcare facility system 109, which includes the aggregator application 165. Meanwhile, the senior healthcare worker and thus, the second device 106 are located external to the healthcare facility 203. In this embodiment, the aggregator application 165 may collect data from the first device 103 and the medical devices 112 using a private network operating within the healthcare facility 203 (since the first devices 103 and medical devices 112 are also within the healthcare facility 203). The aggregator application 165 may transmit the aggregated data (e.g., the medical data 230 including the current patient data 168 and medical device data 170) to the second device 106 over a cellular radio (is “radio cellular” correct? Should it be “cellular radio”?) portion of the communication network 100. Method 300 may be performed after the first device 103 and/or second device 106 have authenticated with the authentication application 181, and after the aggregator application 165 has received the network slice data 176 from the authentication application 181, as described above with reference to FIG. 2A.

The aggregator application 165 may obtain the network slice data 176 and/or the medical data 230, which includes, for example, the current patient data 168 and the medical device data 170. At operation 328, the aggregator application 164 may encrypt the network slice data 176 and/or the medical data 230 using an encryption algorithm programmed at the aggregator application 165 based on the second device identifier 143 of the second device 106. As should be appreciated, different types of encryption algorithms/modules may be used for the encryption and should not be limited herein. However, the encryption key used to perform the encryption may be based on an identifier or identification of the destination of the message/packet, which in this case is the second device identifier 143 (e.g., a SIM identifier of the second device 106). The aggregator application 165 may receive the second device identifier 143 from, for example, the routing application 155, the authentication application 181 of the authentication system 117, or from another data store accessible by the aggregator application 165. In an embodiment, only the medical data 230 is encrypted and added to a payload of a data packet, while the (non-encrypted) network slice data 176 is added to a header of the data packet or added as metadata of the data packet. In this way, network elements in the communication network 100 may use the (non-encrypted) network slice data 176 efficiently to forward the encrypted medical data 230 along a slice-specific path in the network slice 226 identified by the network slice data 176.

At operation 333, the aggregator application 165 may generate one or more data packets comprising the network slice data 176 and the encrypted medical data 230, and then transmit the one or more data packets along the network slice 226 identified by the network slice data 176 using the radio transceiver 161. The second device identifier 143 may be indicated as a destination of the one or more data packets. The network elements in the communication network 100 may be configured to use the network slice data 176 to identify a next hop along a slice-specific path within the network slice 226. The network elements may then forward the one or more data packets comprising the network slice data 176 and the medical data 230 along the path in the network slice 226 until the one or more data packets reach the second device 106.

At operation 335, the second device 106 (e.g., the application 139 at the second device 106) may decrypt the medical data 230 using the first device identifier 135. The second device 106 may, for example, have obtained the first device identifier 135 from a data store accessible by the second device 106 or from the authentication application 181 of the authentication system 117. As described in the AI Communications Application, the senior healthcare worker may use the medical data 230 to generate patient records 172 at the second device 106.

As described in the AI Communications Application, the record application 158 and medical application 160 at the healthcare facility system 109 may also transmit recommendations 140, equipment instructions 145, and medical education data 148 to the second device 106, for the senior healthcare worker to confirm or reject, such that the confirmed recommendations 140, equipment instructions 145, and medical education data 148 may be sent to the first device 103. The AI Communications Application further describes the process of generating and sending the recommendations 140, equipment instructions 145, and medical education data 148 to the second device 106 and receiving confirmations/rejections on the recommendations 140, equipment instructions 145, and medical education data 148. In an embodiment, the recommendations 140, equipment instructions 145, and medical education data 148 may be encrypted using the second device identifier 143 and transmitted from the healthcare facility system 109 to the second device 106 over the network slice 226, in a manner similar to that described above.

The second device 106 may generate patient records 172 and confirm recommendations 140, equipment instructions 145, and medical education data 148. At operation 336, the second device 106 may encrypt the patient records 172 using an identifier of the healthcare facility system 109, and transmit the patient records 172 to the healthcare facility system 109 using the radio transceiver 146. The aggregator application 165 may store the patient records 172 at the data store 162. At operation 336, the second device 106 may also encrypt the recommendations 140, equipment instructions 145, and/or medical education data 148 using the identifier of the healthcare facility system 109. At operation 339, the second device 106 may then transmit the patient records 172, recommendations 140, equipment instructions 145, and medical education data 148 with the network slice data 176 across a slice-specific path in the network slice 226 to the healthcare facility system 109 using the radio transceiver 146. In an embodiment, the second device 106 may encrypt the recommendations 140, equipment instructions 145, and/or medical education data 148 using the first device identifier 135. The second device 106 may then transmit the encrypted recommendations 140, equipment instructions 145, and medical education data 148 within the network slice data 176 across a slice-specific path in the network slice 226 to the first device 103.

At operation 345, the aggregator application 165 may decrypt the patient records 172, recommendations 140, equipment instructions 145, and/or medical education data 148 using the second device identifier 143. When the first device 103 receives the recommendations 140, equipment instructions 145, and medical education data 148, the application 125 at the first device 103 may decrypt the recommendations 140, equipment instructions 145, and/or medical education data 148 using the second device identifier 143. The first device 103 may display the recommendations 140, equipment instructions 145, and medical education data 148 at the display 127 of the first device 103, as described in the AI Communications Application.

FIG. 3B is a block diagram illustrating a method 350 for providing enhanced, network slice-based communications between healthcare workers and patients. In the example shown in FIG. 3B, the first healthcare worker and the patient are located external to a healthcare facility 203 (e.g., at a home of the patient, in some cases in a rural area with less cellular signal strength). Meanwhile, the senior healthcare worker and thus, the second device 106 are located within the healthcare facility 203 (e.g., in an office room inside the healthcare facility 203). In this embodiment, the aggregator application 165 may collect data to be transmitted to the second device 106 and from the second device 106 using a private network operating within the healthcare facility 203 (since the second device 106 is also within the healthcare facility 203). The aggregator application 165 may then communicate data from different sources, such as the first device 103 and medical devices 112, over a radio cellular portion of the communication network 100. Method 350 may be performed after the first device 103 and/or second device 106 have authenticated with the authentication application 181, and after the first device 103 has received the network slice data 176 from the authentication application 181, as described above with reference to FIG. 2A.

The first device 103 (e.g., the application 125) may receive the network slice data 176 from the authentication application 181 and/or receive the medical data 230 from the first healthcare worker (e.g., via the user interface 129) and the medical devices 112. At operation 353, the first device 103 (e.g., the application 125) may encrypt the network slice data 176 and/or the medical data 230 using an encryption algorithm based on an identifier of the healthcare facility system 109 or the second device identifier 143 of the second device 106, depending on the destination of the medical data 230. The first device 103 may receive the identifier of the healthcare facility system 109 or the second device identifier 143 from, for example, the routing application 155, the authentication application 181 of the authentication system 117, or from another data store accessible by the aggregator application 165. In an embodiment, only the medical data 230 is encrypted and added to a payload of a data packet, while the (non-encrypted) network slice data 176 is added to a header of the data packet or added as metadata of the data packet.

At operation 356, the first device 103 (e.g., the application 125) may generate one or more data packets comprising the network slice data 176 and the medical data 230, and then transmit the one or more data packets along the network slice 226 identified by the network slice data 176 using the radio transceiver 130. The second device identifier 143 may be indicated as a destination of the one or more data packets. The network elements in the communication network 100 may be configured to use the network slice data 176 to identify a next hop along a slice-specific path within the network slice 226. The network elements may then forward the one or more data packets comprising the network slice data 176 and the medical data 230 along the path within the network slice 226 until the one or more data packets reach the second device 106.

At operation 359, the aggregator application 165 may decrypt the medical data 230 using the first device identifier 135, and forward the decrypted medical data 230 to the second device 106. The aggregator application 165 may, for example, have obtained the first device identifier 135 from a data store accessible by the second device 106 or from the authentication application 181 of the authentication system 117. As described in the AI Communications Application, the senior healthcare worker may use the medical data 230 to generate patient records 172 at the second device 106.

As described in the AI Communications Application, the record application 158 and medical application 160 at the healthcare facility system 109 may also transmit recommendations 140, equipment instructions 145, and medical education data 148 to the second device 106 via the private network of the healthcare facility 203. The senior healthcare worker may then confirm or reject recommendations 140, equipment instructions 145, and medical education data 148, such that the confirmed recommendations 140, equipment instructions 145, and medical education data 148 may be sent to the first device 103 over the cellular radio network. As such, the second device 106 may generate patient records 172 and confirm recommendations 140, equipment instructions 145, and medical education data 148.

At operation 361, the second device 106 may encrypt the patient records 172 using the first device identifier 135 of the first device 103. The second device 106 may transmit the encrypted patient records 172 to the data store 162 via the private network of the healthcare facility 203. In an embodiment, the second device 106 may encrypt the recommendations 140, equipment instructions 145, and/or medical education data 148 using the first device identifier 135. At operation 364, the second device 106 may transmit the recommendations 140, equipment instructions 145, and medical education data 148 with the network slice data 176 across a slice-specific path in the network slice 226 to the healthcare facility system 109 using the radio transceiver 146.

At operation 367, the first device 103 (e.g., application 125) may decrypt the recommendations 140, equipment instructions 145, and/or medical education data 148 using the second device identifier 143. The first device 103 may display the recommendations 140, equipment instructions 145, and medical education data 148 at the display 127 of the first device 103, as described in the AI Communications Application.

FIG. 3C is a block diagram illustrating a method 375 for providing enhanced, network slice-based communications between healthcare workers and patients. In the example shown in FIG. 3C, the first healthcare worker, the patient, and the second healthcare worker are all external to a healthcare facility. In this embodiment, the first device 103, second device 106, and the applications at the healthcare facility system 109 may communicate over a cellular radio portion of the communication network 100. Method 375 may be performed after the first device 103 and/or second device 106 have authenticated with the authentication application 181, and after the first device 103 has received the network slice data 176 from the authentication application 181, as described above with reference to FIG. 2A.

The first device 103 (e.g., the application 125) may receive the network slice data 176 from the authentication application 181 and/or receive the medical data 230 from the first healthcare worker (e.g., via the user interface 129) and the medical devices 112. At operation 378, the first device 103 (e.g., the application 125) may encrypt the network slice data 176 and/or the medical data 230 using an encryption algorithm based on the second device identifier 143 of the second device 106. In an embodiment, only the medical data 230 is encrypted and added to a payload of a data packet, while the (non-encrypted) network slice data 176 is added to a header of the data packet or added as metadata of the data packet.

At operation 381, the first device 103 (e.g., the application 125) may generate one or more data packets comprising the network slice data 176 and the medical data 230, and then transmit the one or more data packets to the second device 106 along the network slice 226 identified by the network slice data 176 using the radio transceiver 130. The second device identifier 143 may be indicated as a destination of the one or more data packets. The network elements in the communication network 100 may be configured to use the network slice data 176 to identify a next hop along a slice-specific path within the network slice 226. The senior healthcare worker may operate the second device 106 to generate patient records 270, at operation 382.

In an embodiment, the first device 103 may also encrypt the medical data 230 using the identifier of the healthcare facility system 109. As shown in FIG. 3C, at operation 381, the first device 103 may also transmit the one or more data packets including the network slice data 176 and the encrypted medical data 320 to the healthcare facility system 109 along the network slice 226 identified by the network slice data 176 using the radio transceiver 130.

At operation 383, the second device 106 (e.g., the application 139 at the second device 106) may decrypt the medical data 230 using the first device identifier 135. As described in the AI Communications Application, the senior healthcare worker may use the medical data 230 to generate patient records 172 at the second device 106. The record application 158 and medical application 160 at the healthcare facility system 109 may generate recommendations 140, equipment instructions 145, and medical education data 148. At operation 385, the record application 158 and medical application 160 at the healthcare facility system 109 may encrypt the recommendations 140, equipment instructions 145, and medical education data 148 using the second device identifier 143 of the second device 106 based on an encryption algorithm. The record application 158 and the medical application 160 may transmit the recommendations 140, equipment instructions 145, and medical education data 148 to the aggregator application 165 via the private network of the healthcare facility 203. The aggregator application 165 may package the recommendations 140, equipment instructions 145, and medical education data 148 into one or more data packets. At operation 388, the aggregator application 165 may transmit data packets including the recommendations 140, equipment instructions 145, and medical education data 148 to the second device 106 along the network slice 226 identified by the network slice data 176 using the radio transceiver 161.

At operation 390, the second device 106 (e.g., the application 139) may decrypt the recommendations 140, equipment instructions 145, and medical education data 148 received from the healthcare facility system 109 using the identifier of the healthcare facility system 109. The senior healthcare worker may select accurate recommendations 140, equipment instructions 145, and medical education data 148 to confirm the recommendations 140, equipment instructions 145, and medical education data 148 that are to be sent to the first healthcare worker.

At operation 390, the second device 106 (e.g., the application 139) may also encrypt the patient records 172 using the first device identifier 135. At operation 390, the second device 106 may also encrypt the confirmed recommendations 140, equipment instructions 145, and/or medical education data 148 using the first device identifier 135. At operation 391, the second device 106 may transmit the patient records 172 within the network slice data 176 across a slice-specific path in the network slice 226 to the healthcare facility system 109 using the radio transceiver 146. At operation 392, the second device 106 may transmit the patient records 172, and the confirmed recommendations 140, equipment instructions 145, and medical education data 148 within the network slice data 176 across a slice-specific path in the network slice 226 to the first device 103 using the radio transceiver 146. The first device 103 (e.g., application 125) may decrypt the patient records 172, the recommendations 140, equipment instructions 145, and/or medical education data 148 using the second device identifier 143. The first device 103 may display the recommendations 140, equipment instructions 145, and medical education data 148 at the display 127 of the first device 103, as described in the AI Communications Application.

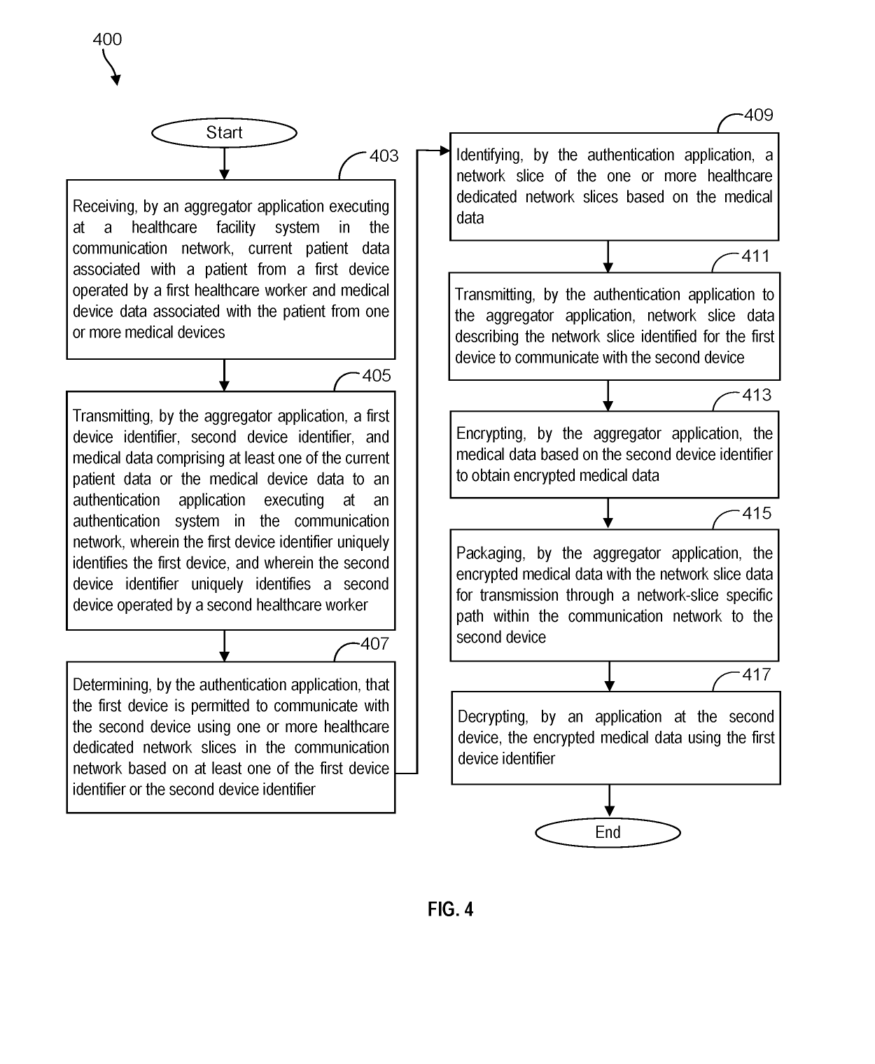

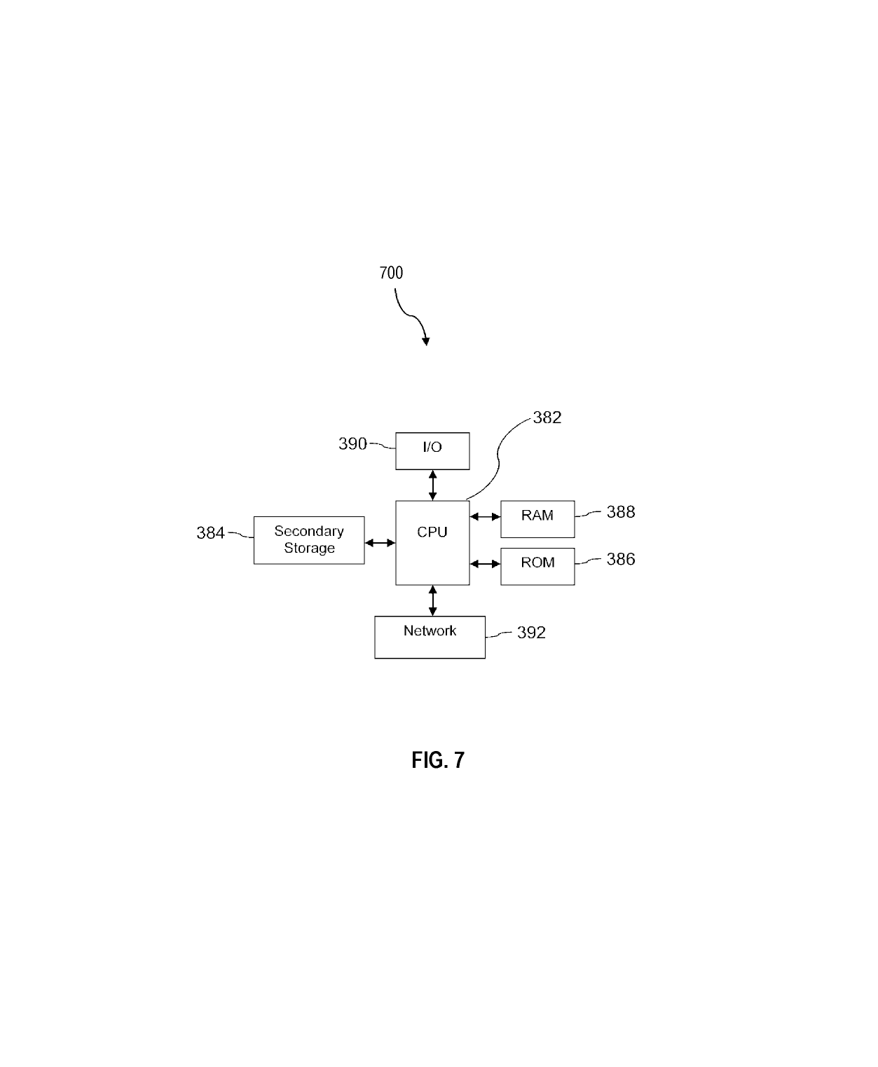

Referring now to FIG. 4, shown is a method 400 for providing enhanced, network slice-based communications between healthcare workers and patients according to various embodiments of the disclosure. Method 400 may be performed by the aggregator application 165 at the healthcare facility system 109, the application 125 at the first device 103, the application 139 at the second device 106, the application 194 at the medical devices 112, and the authentication application 181 at the authentication system 117. Hereinafter, the junior healthcare worker may also be referred to as a “first healthcare worker,” while the senior healthcare worker may also be referred to as a “second healthcare worker.” In embodiments, the method 400 may be implemented using a computer system with components as shown in FIG. 7. As illustrated, method 400 of FIG. 4 includes a number of enumerated operations, but embodiments of the operations in FIG. 4 may include additional operations before, after, and in between the enumerated operations. In some embodiments, one or more of the enumerated operations may be omitted or performed in a different order.

At step 403, method 400 may comprise receiving, by an aggregator application 165 executing at a healthcare facility system 109 in the communication network 100, current patient data 168 associated with a patient from a first device 103 operated by a first healthcare worker and medical device data 170 associated with the patient from one or more medical devices 112. At step 405, method 400 may comprise transmitting, by the aggregator application 165, a first device identifier 135, a second device identifier 143, and medical data 230 comprising at least one of the current patient data 168 or the medical device data 170 to an authentication application 181 executing at an authentication system 117 in the communication network 100. The first device identifier 135 uniquely identifies the first device 103, and the second device identifier 143 uniquely identifies a second device 106 operated by a second healthcare worker.

At step 407, method 400 may comprise determining, by the authentication application 181, that the first device 103 is permitted to communicate with the second device 106 using one or more healthcare-dedicated network slices 226 in the communication network 100 based on at least one of the first device identifier 135 or the second device identifier 143. At step 409, method 400 may comprise identifying, by the authentication application 181, a network slice 226 of the one or more healthcare-dedicated network slices 226 based on the medical data 230.

At step 411, method 400 may comprise transmitting, by the authentication application 181 to the aggregator application 165, network slice data 176 describing the network slice 226 identified for the first device 103 to communicate with the second device 106. At step 413, method 400 may comprise encrypting, by the aggregator application 165, the medical data 230 based on the second device identifier 143 to obtain encrypted medical data 230. At step 415, method 400 may comprise packaging, by the aggregator application 165, the encrypted medical data 230 with the network slice data 176 for transmission through a network-slice specific path within the communication network 100 to the second device 106. At step 417, method 400 may comprise decrypting, by an application 139 at the second device 106, the encrypted medical data 230 using the first device identifier 135.

Method 400 may include other steps and/or features that are not otherwise shown in FIG. 4. In an embodiment, the junior healthcare worker has direct access to the patient while the senior healthcare worker is positioned remote from the patient, and the current patient data 168 indicates current symptoms being experienced by the patient, while the medical device data 170 indicates biometric data associated with the patient. In an embodiment, determining, by the authentication application 181, that the first device 103 is permitted to communicate with the second device 106 using the one or more healthcare-dedicated network slices 226 in the communication network 100 comprises at least one of identifying, by the authentication application 181, the first device identifier 135 as being authorized to use the one or more healthcare-dedicated network slices 226 based on a prior registration of the first device 103, or identifying, by the authentication application 181, the second device identifier 143 as being authorized to use the one or more healthcare-dedicated network slices 226 based on a prior registration of the second device 106.

In an embodiment, the first device identifier 135 is a first SIM identifier of a SIM 131 (e.g., first SIM card or a first eSIM profile) of the first device 103, and the second device identifier 143 is a second SIM identifier of a SIM 144 (e.g., second SIM card or a second eSIM profile of the second device 106). In an embodiment, the first device identifier 135 is a first address of the first device 103, and the second device identifier 143 is an address of the second device 106. In an embodiment, method 400 may further comprise maintaining, in a data store 183 in the communication network 100, the first device identifier 135 in association with identification data describing the first healthcare worker and the second device identifier 143 in association with second identification data describing the second healthcare worker. In an embodiment, network elements in the one or more healthcare-dedicated network slices 226 are permitted to be used for non-healthcare data traffic when healthcare data traffic is not being forwarded through the one or more healthcare-dedicated network slices 226.

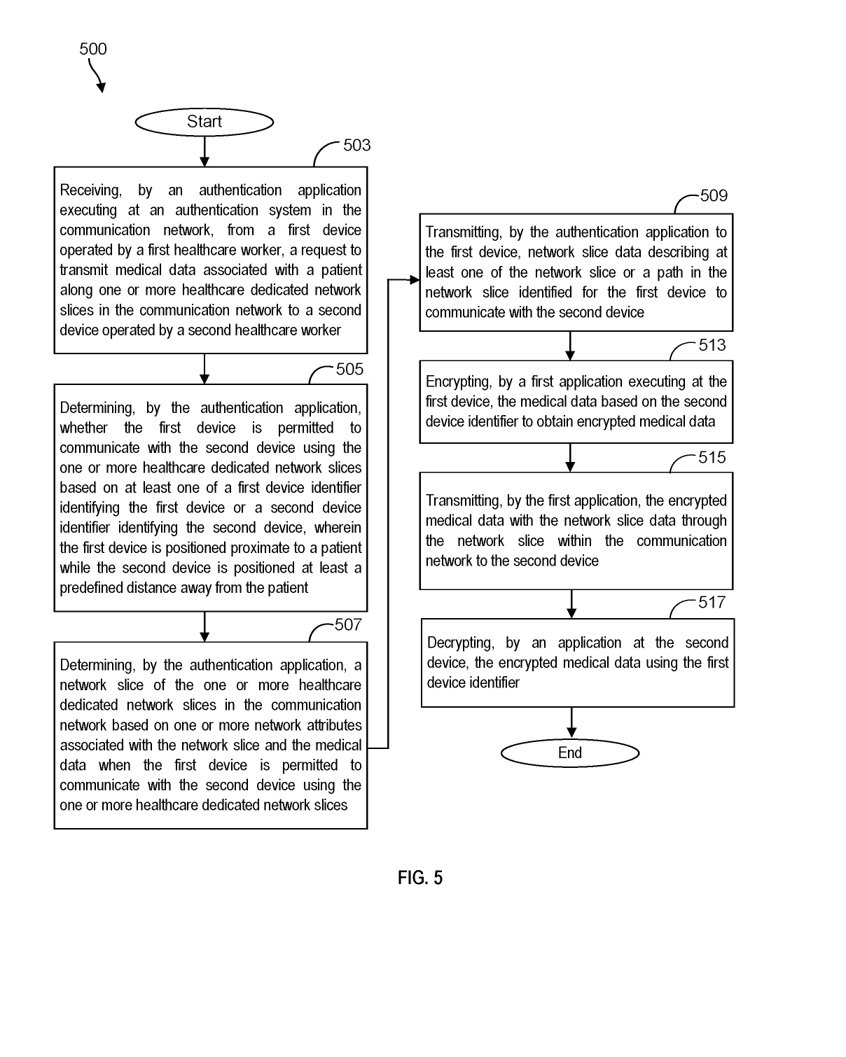

Referring now to FIG. 5, shown is a method 500 for providing enhanced, network slice-based communications between healthcare workers and patients according to various embodiments of the disclosure. Method 500 may be performed by the aggregator application 165 at the healthcare facility system 109, the application 125 at the first device 103, the application 139 at the second device 106, the application 194 at the medical devices 112, and the authentication application 181 at the authentication system 117. Hereinafter, the junior healthcare worker may also be referred to as a “first healthcare worker,” while the senior healthcare worker may also be referred to as a “second healthcare worker.” In embodiments, the method 500 may be implemented using a computer system with components as shown in FIG. 7. As illustrated, method 500 of FIG. 5 includes a number of enumerated operations, but embodiments of the operations in FIG. 5 may include additional operations before, after, and in between the enumerated operations. In some embodiments, one or more of the enumerated operations may be omitted or performed in a different order.

At step 503, method 500 may comprise receiving, by an authentication application 181 executing at an authentication system 117 in the communication network 100, from a first device 103 operated by a first healthcare worker, a request to transmit medical data 230 associated with a patient along one or more healthcare-dedicated network slices 226 in the communication network 100 to a second device 106 operated by a second healthcare worker. At step 505, method 500 may comprise determining, by the authentication application 181, whether the first device 103 is permitted to communicate with the second device 106 using the one or more healthcare-dedicated network slices 226 based on at least one of a first device identifier 135 identifying the first device 103 or a second device identifier 143 identifying the second device 106, the first device 103 being positioned proximate to a patient while the second device 106 is positioned at least a predefined distance away from the patient.

At step 507, method 500 may comprise determining, by the authentication application 181, a network slice 226 of the one or more healthcare-dedicated network slices 226 in the communication network 100 based on one or more network attributes 177 associated with the network slice 226 and the medical data 230 when the first device 103 is permitted to communicate with the second device 106 using the one or more healthcare-dedicated network slices 226. At step 509, method 500 may comprise transmitting, by the authentication application 181 to the first device 103, network slice data 176 describing at least one of the network slice 226 or a path in the network slice 226 identified for the first device 103 to communicate with the second device 106.

At step 513, method 500 may comprise encrypting, by the first application 125 executing at the first device 103, the medical data 230 based on the second device identifier 143 to obtain encrypted medical data 230. At step 515, method 500 may comprise transmitting, by first application 125, the encrypted medical data 230 with the network slice data 176 through the network slice 226 within the communication network 100 to the second device 106. At step 517, method 500 may comprise decrypting, by an application 139 at the second device 106, the encrypted medical data 230 using the first device identifier 135.

Method 500 may include other steps and/or features that are not otherwise shown in FIG. 5. In an embodiment, the medical data 230 comprises current patient data 168 and medical device data 170, and the current patient data 168 indicates current symptoms being experienced by the patient, while the medical device data 170 indicates biometric data associated with the patient. In an embodiment, determining, by the authentication application 181, that the first device 103 is permitted to communicate with the second device 106 using the one or more healthcare-dedicated network slices 226 in the communication network 100 comprises at least one of identifying, by the authentication application 181, the first device identifier 135 as being authorized to use the one or more healthcare-dedicated network slices 226 based on a prior registration of the first device 103, or identifying, by the authentication application 181, the second device identifier 143 as being authorized to use the one or more healthcare-dedicated network slices 226 based on a prior registration of the second device 106.

In an embodiment, the first device identifier 135 is a first SIM identifier of a SIM 131 (e.g., first SIM card or a first eSIM profile) of the first device 103, and the second device identifier 143 is a second SIM identifier of a SIM 144 (e.g., second SIM card or a second eSIM profile of the second device 106). In an embodiment, the first device identifier 135 is a first address of the first device 103, and the second device identifier 143 is an address of the second device 106. In an embodiment, the network slice 226 is provisioned with network elements in the communication network 100 in response to determining, by the authentication application 181, the network slice 226. In an embodiment, the network slice 226 is a pre-provisioned network slice in the communication network 100. In an embodiment, network elements in the one or more healthcare-dedicated network slices 226 are permitted to be used for non-healthcare data traffic when healthcare data traffic is not being forwarded through the one or more healthcare-dedicated network slices 226.

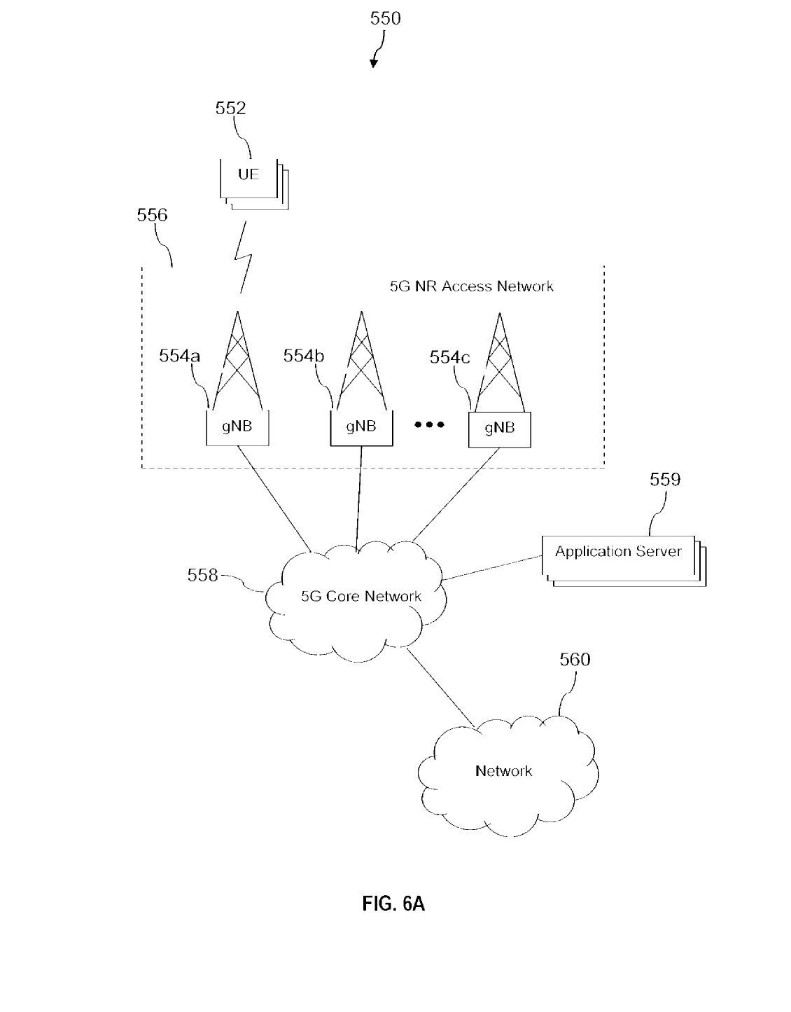

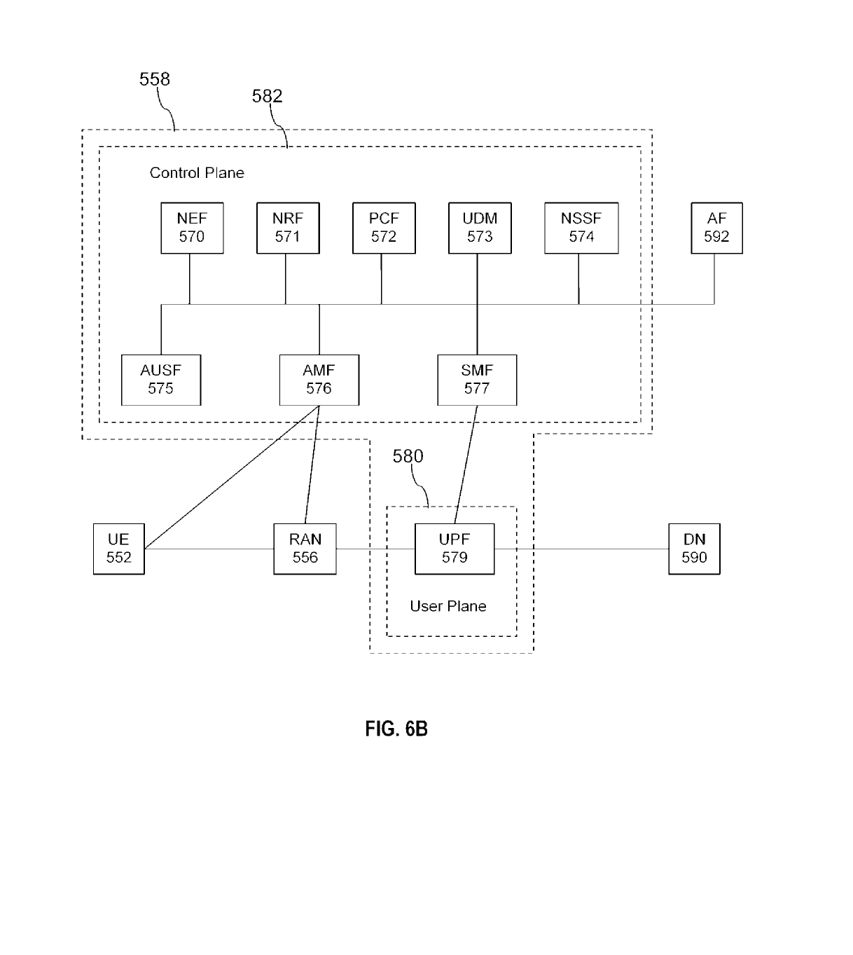

Turning now to FIG. 6A, an exemplary communication system 550 is described. In an embodiment, the communication system 550 may be implemented in the system 100 of FIG. 1. The communication system 550 includes a number of access nodes 554 that are configured to provide coverage in which UEs 552, such as cell phones, tablet computers, machine-type-communication devices, tracking devices, embedded wireless modules, and/or other wirelessly equipped communication devices (whether or not user operated), or devices such as the carrier hotspot device 105, can operate. The access nodes 554 may be said to establish an access network 556. The access network 556 may be referred to as RAN in some contexts. In a 5G technology generation an access node 554 may be referred to as a gigabit Node B (gNB). In 4G technology (e.g., LTE technology) an access node 554 may be referred to as an eNB. In 3G technology (e.g., CDMA and GSM) an access node 554 may be referred to as a base transceiver station (BTS) combined with a base station controller (BSC). In some contexts, the access node 554 may be referred to as a cell site or a cell tower. In some implementations, a picocell may provide some of the functionality of an access node 554, albeit with a constrained coverage area. Each of these different embodiments of an access node 554 may be considered to provide roughly similar functions in the different technology generations.

In an embodiment, the access network 556 comprises a first access node 554a, a second access node 554b, and a third access node 554c. It is understood that the access network 556 may include any number of access nodes 554. Further, each access node 554 could be coupled with a core network 558 that provides connectivity with various application servers 559 and/or a network 560. In an embodiment, at least some of the application servers 559 may be located close to the network edge (e.g., geographically close to the UE 552 and the end user) to deliver so-called “edge computing.” The network 560 may be one or more private networks, one or more public networks, or a combination thereof. The network 560 may comprise the public switched telephone network (PSTN). The network 560 may comprise the Internet. With this arrangement, a UE 552 within coverage of the access network 556 could engage in air-interface communication with an access node 554 and could thereby communicate via the access node 554 with various application servers and other entities.

The communication system 550 could operate in accordance with a particular radio access technology (RAT), with communications from an access node 554 to UEs 552 defining a downlink or forward link and communications from the UEs 552 to the access node 554 defining an uplink or reverse link. Over the years, the industry has developed various generations of RATs, in a continuous effort to increase available data rate and quality of service for end users. These generations have ranged from “1G,” which used simple analog frequency modulation to facilitate basic voice-call service, to “4G” – such as Long Term Evolution (LTE), which now facilitates mobile broadband service using technologies such as orthogonal frequency division multiplexing (OFDM) and multiple input multiple output (MIMO).

Recently, the industry has been exploring developments in “5G” and particularly “5G NR” (5G New Radio), which may use a scalable OFDM air interface, advanced channel coding, massive MIMO, beamforming, mobile mmWave (e.g., frequency bands above 24 GHz), and/or other features, to support higher data rates and countless applications, such as mission-critical services, enhanced mobile broadband, and massive Internet of Things (IoT). 5G is hoped to provide virtually unlimited bandwidth on demand, for example providing access on demand to as much as 20 gigabits per second (Gbps) downlink data throughput and as much as 10 Gbps uplink data throughput. Due to the increased bandwidth associated with 5G, it is expected that the new networks will serve, in addition to conventional cell phones, general internet service providers for laptops and desktop computers, competing with existing ISPs such as cable internet, and also will make possible new applications in internet of things (IoT) and machine to machine areas.

In accordance with the RAT, each access node 554 could provide service on one or more radio-frequency (RF) carriers, each of which could be frequency division duplex (FDD), with separate frequency channels for downlink and uplink communication, or time division duplex (TDD), with a single frequency channel multiplexed over time between downlink and uplink use. Each such frequency channel could be defined as a specific range of frequency (e.g., in radio-frequency (RF) spectrum) having a bandwidth and a center frequency and thus extending from a low-end frequency to a high-end frequency. Further, on the downlink and uplink channels, the coverage of each access node 554 could define an air interface configured in a specific manner to define physical resources for carrying information wirelessly between the access node 554 and UEs 552.

Without limitation, for instance, the air interface could be divided over time into frames, subframes, and symbol time segments, and over frequency into subcarriers that could be modulated to carry data. The example air interface could thus define an array of time-frequency resource elements each being at a respective symbol time segment and subcarrier, and the subcarrier of each resource element could be modulated to carry data. Further, in each subframe or other transmission time interval (TTI), the resource elements on the downlink and uplink could be grouped to define physical resource blocks (PRBs) that the access node could allocate as needed to carry data between the access node and served UEs 552.

In addition, certain resource elements on the example air interface could be reserved for special purposes. For instance, on the downlink, certain resource elements could be reserved to carry synchronization signals that UEs 552 could detect as an indication of the presence of coverage and to establish frame timing, other resource elements could be reserved to carry a reference signal that UEs 552 could measure in order to determine coverage strength, and still other resource elements could be reserved to carry other control signaling such as PRB-scheduling directives and acknowledgement messaging from the access node 554 to served UEs 552. And on the uplink, certain resource elements could be reserved to carry random access signaling from UEs 552 to the access node 554, and other resource elements could be reserved to carry other control signaling such as PRB-scheduling requests and acknowledgement signaling from UEs 552 to the access node 554.

The access node 554, in some instances, may be split functionally into a radio unit (RU), a distributed unit (DU), and a central unit (CU) where each of the RU, DU, and CU have distinctive roles to play in the access network 556. The RU provides radio functions. The DU provides L1 and L2 real-time scheduling functions; and the CU provides higher L2 and L3 non-real time scheduling. This split supports flexibility in deploying the DU and CU. The CU may be hosted in a regional cloud data center. The DU may be co-located with the RU, or the DU may be hosted in an edge cloud data center.

Turning now to FIG. 6B, further details of the core network 558 are described. In an embodiment, the core network 558 is a 5G core network. 5G core network technology is based on a service based architecture paradigm. Rather than constructing the 5G core network as a series of special purpose communication nodes (e.g., an HSS node, an MME node, etc.) running on dedicated server computers, the 5G core network is provided as a set of services or network functions. These services or network functions can be executed on virtual servers in a cloud computing environment which supports dynamic scaling and avoidance of long-term capital expenditures (fees for use may substitute for capital expenditures). These network functions can include, for example, a user plane function (UPF) 579, an authentication server function (AUSF) 575, an access and mobility management function (AMF) 576, a session management function (SMF) 577, a network exposure function (NEF) 570, a network repository function (NRF) 571, a policy control function (PCF) 572, a unified data management (UDM) 573, a network slice selection function (NSSF) 574, and other network functions. The network functions may be referred to as virtual network functions (VNFs) in some contexts.

Network functions may be formed by a combination of small pieces of software called microservices. Some microservices can be re-used in composing different network functions, thereby leveraging the utility of such microservices. Network functions may offer services to other network functions by extending application programming interfaces (APIs) to those other network functions that call their services via the APIs. The 5G core network 558 may be segregated into a user plane 580 and a control plane 582, thereby promoting independent scalability, evolution, and flexible deployment.

The UPF 579 delivers packet processing and links the UE 552, via the access network 556, to a data network 590 (e.g., the network 560 illustrated in FIG. 6A). The AMF 576 handles registration and connection management of non-access stratum (NAS) signaling with the UE 552. Said in other words, the AMF 576 manages UE registration and mobility issues. The AMF 576 manages reachability of the UEs 552 as well as various security issues. The SMF 577 handles session management issues. Specifically, the SMF 577 creates, updates, and removes (destroys) protocol data unit (PDU) sessions and manages the session context within the UPF 579. The SMF 577 decouples other control plane functions from user plane functions by performing dynamic host configuration protocol (DHCP) functions and IP address management functions. The AUSF 575 facilitates security processes.

The NEF 570 securely exposes the services and capabilities provided by network functions. The NRF 571 supports service registration by network functions and discovery of network functions by other network functions. The PCF 572 supports policy control decisions and flow based charging control. The UDM 573 manages network user data and can be paired with a user data repository (UDR) that stores user data such as customer profile information, customer authentication number, and encryption keys for the information. An application function 592, which may be located outside of the core network 558, exposes the application layer for interacting with the core network 558. In an embodiment, the application function 592 may be executed on an application server 559 located geographically proximate to the UE 552 in an “edge computing” deployment mode. The core network 558 can provide a network slice to a subscriber, for example an enterprise customer, that is composed of a plurality of 5G network functions that are configured to provide customized communication service for that subscriber, for example to provide communication service in accordance with communication policies defined by the customer. The NSSF 574 can help the AMF 576 to select the network slice instance (NSI) for use with the UE 552.

FIG. 7 illustrates a computer system 700 suitable for implementing one or more embodiments disclosed herein. In an embodiment, first devices 103, second device 106, AI model 115, medical devices 112, and/or healthcare facility system 109 may each be implemented as the computer system 700. The computer system 700 includes a processor 382 (which may be referred to as a central processor unit or CPU) that is in communication with memory devices including secondary storage 384, read only memory (ROM) 386, random access memory (RAM) 388, input/output (I/O) devices 390, and network connectivity devices 392. The processor 382 may be implemented as one or more CPU chips.

It is understood that by programming and/or loading executable instructions onto the computer system 700, at least one of the CPU 382, the RAM 388, and the ROM 386 are changed, transforming the computer system 700 in part into a particular machine or apparatus having the novel functionality taught by the present disclosure. It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the system 700 is turned on or booted, the CPU 382 may execute a computer program or application. For example, the CPU 382 may execute software or firmware stored in the ROM 386 or stored in the RAM 388. In some cases, on boot and/or when the application is initiated, the CPU 382 may copy the application or portions of the application from the secondary storage 384 to the RAM 388 or to memory space within the CPU 382 itself, and the CPU 382 may then execute instructions that the application is comprised of. In some cases, the CPU 382 may copy the application or portions of the application from memory accessed via the network connectivity devices 392 or via the I/O devices 390 to the RAM 388 or to memory space within the CPU 382, and the CPU 382 may then execute instructions that the application is comprised of. During execution, an application may load instructions into the CPU 382, for example load some of the instructions of the application into a cache of the CPU 382. In some contexts, an application that is executed may be said to configure the CPU 382 to do something, e.g., to configure the CPU 382 to perform the function or functions promoted by the subject application. When the CPU 382 is configured in this way by the application, the CPU 382 becomes a specific purpose computer or a specific purpose machine.

The secondary storage 384 is typically comprised of one or more disk drives or tape drives and is used for non-volatile storage of data and as an over-flow data storage device if RAM 388 is not large enough to hold all working data. Secondary storage 384 may be used to store programs which are loaded into RAM 388 when such programs are selected for execution. The ROM 386 is used to store instructions and perhaps data which are read during program execution. ROM 386 is a non-volatile memory device which typically has a small memory capacity relative to the larger memory capacity of secondary storage 384. The RAM 388 is used to store volatile data and perhaps to store instructions. Access to both ROM 386 and RAM 388 is typically faster than to secondary storage 384. The secondary storage 384, the RAM 388, and/or the ROM 386 may be referred to in some contexts as computer readable storage media and/or non-transitory computer readable media.

I/O devices 390 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 392 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards, and/or other well-known network devices. The network connectivity devices 392 may provide wired communication links and/or wireless communication links (e.g., a first network connectivity device 392 may provide a wired communication link and a second network connectivity device 392 may provide a wireless communication link). Wired communication links may be provided in accordance with Ethernet (IEEE 802.3), Internet protocol (IP), time division multiplex (TDM), data over cable service interface specification (DOCSIS), wavelength division multiplexing (WDM), and/or the like. In an embodiment, the radio transceiver cards may provide wireless communication links using protocols such as code division multiple access (CDMA), global system for mobile communications (GSM), long-term evolution (LTE), WiFi (IEEE 802.11), Bluetooth, Zigbee, narrowband Internet of things (NB IoT), near field communications (NFC), and radio frequency identity (RFID). The radio transceiver cards may promote radio communications using 5G, 5G New Radio, or 5G LTE radio communication protocols. These network connectivity devices 392 may enable the processor 382 to communicate with the Internet or one or more intranets. With such a network connection, it is contemplated that the processor 382 might receive information from the network, or might output information to the network in the course of performing the above-described method steps. Such information, which is often represented as a sequence of instructions to be executed using processor 382, may be received from and outputted to the network, for example, in the form of a computer data signal embodied in a carrier wave.

Such information, which may include data or instructions to be executed using processor 382 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several methods well-known to one skilled in the art. The baseband signal and/or signal embedded in the carrier wave may be referred to in some contexts as a transitory signal.

The processor 382 executes instructions, codes, computer programs, scripts which it accesses from hard disk, floppy disk, optical disk (these various disk based systems may all be considered secondary storage 384), flash drive, ROM 386, RAM 388, or the network connectivity devices 392. While only one processor 382 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 384, for example, hard drives, floppy disks, optical disks, and/or other device, the ROM 386, and/or the RAM 388 may be referred to in some contexts as non-transitory instructions and/or non-transitory information.

In an embodiment, the computer system 700 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 700 to provide the functionality of a number of servers that is not directly bound to the number of computers in the computer system 700. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid state memory chip, for example analog magnetic tape, compact disk read only memory (CD-ROM) disks, floppy disks, jump drives, digital cards, multimedia cards, and others. The computer program product may be suitable for loading, by the computer system 700, at least portions of the contents of the computer program product to the secondary storage 384, to the ROM 386, to the RAM 388, and/or to other non-volatile memory and volatile memory of the computer system 700. The processor 382 may process the executable instructions and/or data structures in part by directly accessing the computer program product, for example by reading from a CD-ROM disk inserted into a disk drive peripheral of the computer system 700. Alternatively, the processor 382 may process the executable instructions and/or data structures by remotely accessing the computer program product, for example by downloading the executable instructions and/or data structures from a remote server through the network connectivity devices 392. The computer program product may comprise instructions that promote the loading and/or copying of data, data structures, files, and/or executable instructions to the secondary storage 384, to the ROM 386, to the RAM 388, and/or to other non-volatile memory and volatile memory of the computer system 700.

In some contexts, the secondary storage 384, the ROM 386, and the RAM 388 may be referred to as a non-transitory computer readable medium or a computer readable storage media. A dynamic RAM embodiment of the RAM 388, likewise, may be referred to as a non-transitory computer readable medium in that while the dynamic RAM receives electrical power and is operated in accordance with its design, for example during a period of time during which the computer system 700 is turned on and operational, the dynamic RAM stores information that is written to it. Similarly, the processor 382 may comprise an internal RAM, an internal ROM, a cache memory, and/or other internal non-transitory storage blocks, sections, or components that may be referred to in some contexts as non-transitory computer readable media or computer readable storage media.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A method implemented in a communication network to provide artificial intelligence enhanced communications between healthcare workers for patient care and documentation, wherein the method comprises:
receiving, by an authentication application executing at an authentication system in the communication network, from a first device operated by a first healthcare worker, a request to transmit medical data associated with a patient along one or more healthcare-dedicated network slices in the communication network to a second device operated by a second healthcare worker,
determining, by the authentication application, whether the first device is permitted to communicate with the second device using the one or more healthcare-dedicated network slices based on at least one of a first device identifier identifying the first device or a second device identifier identifying the second device, wherein the first device is positioned proximate to a patient while the second device is positioned at least a predefined distance away from the patient;
determining, by the authentication application, a network slice of the one or more healthcare-dedicated network slices in the communication network based on one or more network attributes associated with the network slice and the medical data when the first device is permitted to communicate with the second device using the one or more healthcare-dedicated network slices;
transmitting, by the authentication application to the first device, network slice data describing at least one of the network slice or a path in the network slice identified for the first device to communicate with the second device;
encrypting, by a first application executing at the first device, the medical data based on the second device identifier to obtain encrypted medical data;
transmitting, by the first application, the encrypted medical data with the network slice data through the network slice within the communication network to the second device; and
decrypting, by a second application executing at the second device, the encrypted medical data using the first device identifier.

2. The method of claim 1, wherein the medical data comprises patient data and medical device data, wherein the patient data indicates current symptoms being experienced by the patient, and wherein the medical device data indicates biometric data associated with the patient.

3. The method of claim 1, wherein determining, by the authentication application, that the first device is permitted to communicate with the second device using the one or more healthcare-dedicated network slices in the communication network comprises at least one of:
identifying, by the authentication application, the first device identifier as being authorized to use the one or more healthcare-dedicated network slices based on a prior registration of the first device; or
identifying, by the authentication application, the second device identifier as being authorized to use the one or more healthcare-dedicated network slices based on a prior registration of the second device.

4. The method of claim 1, wherein the first device identifier is a first Subscriber Identity Module (SIM) identifier of a first SIM card or a first electronic SIM (eSIM) profile of the first device, and wherein the second device identifier is a second SIM identifier of a second SIM card or a second eSIM profile of the second device.

5. The method of claim 1, wherein the first device identifier is a first address of the first device, and wherein the second device identifier is an address of the second device.

6. The method of claim 1, wherein the network slice is provisioned with network elements in the communication network in response to determining, by the authentication application, the network slice.

7. The method of claim 1, wherein the network slice is a pre-provisioned network slice in the communication network.

8. The method of claim 1, wherein network elements in the one or more healthcare-dedicated network slices are permitted to be used for non-healthcare data traffic when healthcare data traffic is not being forwarded through the one or more healthcare-dedicated network slices.

9. A method implemented in a communication network to provide enhanced, network slice-based communications between healthcare workers for patient care and documentation, wherein the method comprises:
receiving, by an aggregator application executing at a healthcare facility system in the communication network, current patient data associated with a patient from a first device operated by a first healthcare worker and medical device data associated with the patient from one or more medical devices;
transmitting, by the aggregator application, a first device identifier, second device identifier, and medical data comprising at least one of the current patient data or the medical device data to an authentication application executing at an authentication system in the communication network, wherein the first device identifier uniquely identifies the first device, and wherein the second device identifier uniquely identifies a second device operated by a second healthcare worker;
determining, by the authentication application, that the first device is permitted to communicate with the second device using one or more healthcare-dedicated network slices in the communication network based on at least one of the first device identifier or the second device identifier;
identifying, by the authentication application, a network slice of the one or more healthcare-dedicated network slices based on the medical data;
transmitting, by the authentication application to the aggregator application, network slice data describing the network slice identified for the first device to communicate with the second device;
encrypting, by the aggregator application, the medical data based on the second device identifier to obtain encrypted medical data;
packaging, by the aggregator application, the encrypted medical data with the network slice data for transmission through a network-slice specific path within the communication network to the second device; and
decrypting, by an application at the second device, the encrypted medical data using the first device identifier.

10. The method of claim 9, wherein the first healthcare worker has direct access to the patient while the second healthcare worker is positioned remotely from the patient, wherein the current patient data indicates current symptoms being experienced by the patient, and wherein the medical device data indicates biometric data associated with the patient.

11. The method of claim 9, wherein determining, by the authentication application, that the first device is permitted to communicate with the second device using the one or more healthcare-dedicated network slices in the communication network comprises at least one of:
identifying, by the authentication application, the first device identifier as being authorized to use the one or more healthcare-dedicated network slices based on a prior registration of the first device; or
identifying, by the authentication application, the second device identifier as being authorized to use the one or more healthcare-dedicated network slices based on a prior registration of the second device.

12. The method of claim 9, wherein the first device identifier is a first Subscriber Identity Module (SIM) identifier of a first SIM card or a first electronic SIM (eSIM) profile of the first device, and wherein the second device identifier is a second SIM identifier of a second SIM card or a second eSIM profile of the second device.

13. The method of claim 9, wherein the first device identifier is a first address of the first device, and wherein the second device identifier is an address of the second device.

14. The method of claim 9, further comprising maintaining, in a data store in the communication network, the first device identifier in association with identification data describing the first healthcare worker and the second device identifier in association with second identification data describing the second healthcare worker.

15. The method of claim 9, wherein network elements in the one or more healthcare-dedicated network slices are permitted to be used for non-healthcare data traffic when healthcare data traffic is not being forwarded through the one or more healthcare-dedicated network slices.

16. A system, comprising:
a non-transitory memory;
a processor coupled to the non-transitory memory; and
an authentication application stored at the non-transitory memory, which when executed by the processor, causes the processor to be configured to:
receive, from a first device operated by a first healthcare worker, a request to transmit medical data associated with a patient along one or more healthcare-dedicated network slices in a communication network to a second device operated by a second healthcare worker,
determine whether the first device is permitted to communicate with the second device using the one or more healthcare-dedicated network slices based on at least one of a first device identifier identifying the first device or a second device identifier identifying the second device, wherein the first device is positioned proximate to a patient while the second device is positioned at least a predefined distance away from the patient;
determine, based on a network slice policy, a network slice of the one or more healthcare-dedicated network slices when the first device is permitted to communicate with the second device using the one or more healthcare-dedicated network slices, wherein the network slice policy indicates that the medical data is to be transmitted using the network slice based on one or more network attributes associated with the network slice; and
transmit, to the first device, network slice data describing at least one of the network slice or a path in the network slice identified for the first device to communicate with the second device.

17. The system of claim 16, wherein the authentication application further causes the processor to be configured to identify the first device identifier as being authorized to use the one or more healthcare-dedicated network slices based on a prior registration of the first device.

18. The system of claim 16, wherein the authentication application further causes the processor to be configured to identify the second device identifier as being authorized to use the one or more healthcare-dedicated network slices based on a prior registration of the second device.

19. The system of claim 16, wherein the authentication application further causes the processor to be configured to:
determine a first type of data included in the medical data;
identify network requirements associated with the first type of data based on the network slice policy; and
determine the network slice of the one or more healthcare-dedicated network slices based on the network slice including resources that satisfy the network requirements associated with the first type of data.

20. The system of claim 16, wherein the network slice is determined based on a network profile including the network slice data.
